(19) United States
(12) Patent Application Publication
(10) Pub. No.: US 2023/0148432 A1
(43) Pub. Date: May 11, 2023

(54) USE OF STEM CELLS FOR TREATMENT OF EXCESSIVE INFLAMMATION

(71) Applicants: Children's Medical Center Corporation, Boston, MA (US); TICEBA GmbH, Heidelberg (DE)

(72) Inventors: Mark Andreas Kluth, Heidelberg (DE); Markus H. Frank, Cambridge, MA (US); Christoph Ganss, Heidelberg (DE)

(73) Assignees: Children's Medical Center Corporation, Boston, MA (US); TICEBA GmbH, Heidelberg (DE)

(21) Appl. No.: 17/915,364

(22) PCT Filed: Mar. 30, 2021
(86) PCT No.: PCT/US2021/024947

§ 371 Date: Sep. 28, 2022

Related U.S. Application Data
(60) Provisional application No. 63/002,274, filed on Mar. 30, 2020.

(51) Int. Cl. A61K 35/28; A61K 47/46; A61P 29/00; A61P 31/14

(52) U.S. Cl. CPC A61K 35/28; A61K 47/46; A61P 29/00; A61P 31/14

(57) ABSTRACT

Methods and compositions comprising ABCB5+ stem cell to treat hyper-inflammatory disorders, such as acute respiratory distress syndrome (ARDS) are provided. Such compositions may be used to treat, for example, patients with severe COVID19.

### RELATED APPLICATIONS

This application claims priority under 35 U.S.C. § 119(e) to U.S. Provisional Application Ser. No. 63/002,274, filed on March 30, 2020, which is herein incorporated by reference in its entirety.

### BACKGROUND OF INVENTION

The severe acute respiratory syndrome corona virus 2 (SARS-CoV-2), the etiologic factor of coronavirus disease 2019 (COVID-19), has rapidly spread from its origin in Wuhan City of Hubei Province of China to the rest of the world (Singhal 2020). The clinical spectrum of COVID-19 varies from asymptomatic or paucisymptomatic forms to clinical conditions characterized by respiratory failure that necessitates mechanical ventilation and support in an intensive care unit (ICU), to multiorgan and systemic manifestations in terms of sepsis, septic shock, and multiple organ dysfunction syndromes (MODS) (Cascella, Rajnik, and Cuomo 2020). The common clinical features of COVID-19 include cough, sore throat, fever (not in all patients), headache, fatigue, myalgia and breathlessness, making it difficult to distinguish from other respiratory infections. Complications witnessed include acute lung injury, shock, acute kidney injury, liver injury, gastrointestinal symptoms, and acute respiratory distress syndrome (ARDS), which represents the leading cause of mortality (Singhal 2020; Rothan and Byrareddy 2020; Mehta et al. 2020; Xu et al. 2020). The median time from onset of symptoms to dyspnea is 5 days, hospitalization 7 days and ARDS 8 days (Singhal 2020; Mehta et al. 2020). Adverse outcomes and death are more common in the elderly and those with underlying co-morbidities (50-75% of fatal cases) (Singhal 2020).

SARSCoV-2 infection can be roughly divided into three stages: stage I, an asymptomatic incubation period with or without detectable virus; stage II, non-severe symptomatic period with the presence of virus; stage III, severe respiratory symptomatic stage with high viral load. Clinically, the immune responses induced by SARS-CoV-2 infection are two phased. During the incubation and non-severe stages, a specific adaptive immune response is required to eliminate the virus and to preclude disease progression to severe stages (Shi et al. 2020). However, when a protective immune response is impaired, virus will propagate and massive destruction of the affected tissues will occur, especially in organs that have high ACE2 expression, the virus entry receptor, such as lungs, arteries, heart, kidney, and intestines (Shi et al. 2020; Hamming et al. 2004). The damaged cells induce innate inflammation in the lungs that is largely mediated by proinflammatory macrophages and granulocytes. Lung inflammation is the main cause of life-threatening respiratory disorders at the severe stage (Shi et al. 2020). In some cases, chest CT scan show multiple peripheral ground-glass opacities in subpleural regions of both lungs that likely induced both systemic and localized immune response that led to increased inflammation. In addition, based on results from chest radiographs upon admission, some of the cases show an infiltrate in the upper lobe of the lung that is associated with increasing dyspnea with hypoxemia (Rothan and Byrareddy 2020). Once severe lung damage occurs, efforts should be made to suppress inflammation and to manage the symptoms. Alarmingly, after discharge from hospital, some patients remain/return viral positive and others even relapse. This indicates that a virus-eliminating immune response to SARS-CoV-2 may be difficult to induce at least in some patients and vaccines may not work in these individuals (Shi et al. 2020).

### SUMMARY OF THE INVENTION

In some aspect, a method of treating a hyper-inflammatory disorder in a human subject, the method by administering to the subject a composition comprising ABCB5⁺ stem cells in an effective amount to treat the hyper-inflammatory disorder is provided.

In some embodiments the dose is 1×10⁶ to 1×10¹⁰, optionally 1×10⁸ ABCB5⁺ stem cells. In some embodiments the method involves administering the dose to the subject two times. In some embodiments the dose is administered to the subject three times. In some embodiments the dose is administered to the subject four times. In some embodiments the doses are administered one day apart.

In some embodiments the composition comprises ABCB5+ stem cells and a pharmaceutically acceptable excipient. In some embodiments the pharmaceutically acceptable excipient is human serum albumin/Ringer/glucose solution (HRG).

In some embodiments the inflammatory disorder is acute respiratory distress syndrome (ARDS). In some embodiments the subject has a severe COVID-19 infection. In some embodiments administration of the dose increases the level of IL-1RA, IL-10, or both, in the subject. In some embodiments administration of the dose decreases the level of TNF-α, IL-1β, or both, in the subject. In some embodiments administration of the dose promotes a switch from M1 macrophages to M2 macrophages.

In other aspects a method of treating a human subject having a SARS infection is provided. The method comprises administering a composition of ABCB5+ stem cells to the subject in an effective amount to treat the subject. In some embodiments the SARS infection is a SARS-CoV-2 infection.

In some embodiments the ABCB5+ stem cells are dermal ABCB5+ stem cells. In some embodiments the ABCB5+ stem cells are ocular ABCB5+ stem cells. In some embodiments the ABCB5+ stem cells are a population of synthetic ABCB5+ stem cells. In some embodiments greater than 99%, 99.5%, 99.7%, 99.9%, 99.99%, 99.998%, 99.999%, or 99.999997% of the population of synthetic ABCB5+ stem cells are an in vitro progeny of physiologically occurring skin-derived ABCB5-positive mesenchymal stem cells.

In some embodiments the cells are administered intravenously.

In some embodiments a dose of the cells is 1×10⁶ to 1×10¹⁰ ABCB5+ stem cells.

In some embodiments administration of the cells increases the level of IL-1RA, IL-10, or both, in the subject. In some embodiments administration of the cells decreases the level of TNF-α, IL-1β, or both, in the subject. In some embodiments administration of the cells promotes a switch from M1 macrophages to M2 macrophages.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1: In situ characterization of ABCB5-positive cells in their endogenous niche in healthy human skin. Microphotographs of 5 μm sections from healthy human skin subjected to 8 immunostaining for ABCB and the endothelial marker CD31 revealed both a perivascular and a dispersed interfollicular dermal localization of ABCB5-positive cells. ABCB5-positive cells occurred at an average percentage of 2.45±0.61 of all dermal cells as determined in 8-10 microscopic fields of skin sections from 10 different donors. ABCB5+ cells were more abundant in a perivascular localization in the interfollicular dermis compared to a non-perivascular localization. Double immunofluorescence staining for ABCB5 and the pericyte marker NG2 showed that perivascular ABCB5-positive cells are distinct from NG2+ pericytes. A clear co-localization of ABCB5 with the stem cell marker SSEA-4 also was observed in a distinct subpopulation of dermal cells.

FIGS. 2A-2H: In vitro characterization of ABCB5-positive dermal cells. Flow cytometry reproducibly confirmed high purity of ABCB5-positive cells and ABCB5-negative dermal cell fractions (FIG. 2A). Differential interference contrast micrographs depicted a fibroblast-like phenotype for both ABCB5-positive and ABCB5-negative fractions. Flow cytometry results showed that both fractions expressed CD90, CD73 and CD105 and lacked CD14, CD20, CD34 and CD45 expression (FIG. 2B, black represents expression of labeled marker, grey histograms represent isotope controls). In vitro trilineage differentiation was more prominent for ABCB5-positive cells than for ABCB5-depleted dermal cells (FIG. 2C-2E). Adipogenic and osteogenic differentiation was quantified by extraction of respective ORO (FIG. 2C) and ARS (FIG. 2D) indicator dyes. Chondrogenic differentiation (FIG. 2E) was visualized by aggrecan immunostaining and quantitatively assessed by extraction of sulphated glycosaminoglycans (sGAG). Following low-density seeding, only cells from ABCB5-sorted fractions formed crystal violet-visualized colonies (FIG. 2F). ABCB5-positive sorted cell clonal cultures were subjected to a second colony forming unit (CFU) assay and trilineage (O=osteogenic; A=adipogenic; C=chondrogenic) differentiation (FIG. 2G). Threshold for clonogenic growth (five colonies) and positive differentiation was set three standard deviations above the average from ABCB5-negative samples or unstimulated controls, respectively. SOX2+ nuclei by immunofluorescence staining and SSEA-4+ cells by flow cytometry were found exclusively within ABCB5-positive fractions (FIG. 2H).

FIG. 3: Mean number of human cells. Mean number of human cells in skin (at the injection site), skeletal muscle (at the injection site), and lung tissue at different time points. Error bars: mean SD; Statistical analysis: non-paired one-way ANOVA followed by Tukey's multiple comparison test.

FIG. 4: Comparison of body weight development. NSG mice were injected 3 times with ABCB5-positive MSCs or vehicle and body weight measured every week until week 13. Left: males; Right females.

### DETAILED DESCRIPTION OF THE INVENTION

Systemic inflammation seems to be a hallmark of COVID19 patients and a predictor of mortality in affected patients Lancet (Mehta et al. 2020; Huang et al. 2020). Mesenchymal stem cells (MSCs) are known to interact with the inflammatory environment (Hoogduijn et al. 2010; Wada, Gronthos, and Bartold 2013; Wang et al. 2014).

Recent studies on COVID-19 have shown that the incidence of liver injury ranged from 14.8%-53%, mainly indicated by abnormal ALT/AST levels accompanied by slightly elevated bilirubin and decreased albumin levels. The proportion of developing liver injury in severe COVID-19 patients was significantly higher than that in mild patients. In death cases of COVID-19, the incidence of liver injury might reach as high as 58.06% and 78%. It has been shown that ACE2 is expressed in liver cells and, to a greater extent, in bile duct cells, which are known to play important roles in liver regeneration and immune response. Currently, studies on the mechanisms of SARS-CoV-2 related liver injury are limited (Xu et al. 2020). In addition to liver injuries, some articles have also reported an increased incidence of acute kidney injury (AKI) following COVID-19. Noteworthy, these patients have a higher mortality rate compared to other patients who do not develop AKI (Rismanbaf and Zarei 2020).

There is currently no definitive cure for COVID-19 and medicines currently prescribed to treat the disease (Oseltamivir, Lopinavir/Ritonavir, Ribavirin, and Chloroquine Phosphate or Hydroxy Chloroquine Sulfate) are metabolized in the liver. Most of the metabolites derived from these medicines are found in the urine due to renal excretion. Therefore, injury to the liver and kidneys can impair metabolism, excretion, dosing and expected concentrations of the medications, which can increase the risk of toxicity and adverse events. As a result, frequent and careful monitoring of liver and kidney functions in patients with COVID-19 can lead to early diagnosis of liver and kidney disorders, and also help in achieving the optimal therapeutic concentrations and reducing the risk of adverse drug reactions (Rismanbaf and Zarei 2020).

Accumulating evidence suggests that a subgroup of patients with severe COVID-19 might have a cytokine storm syndrome, as recently published in The Lancet (Mehta et al. 2020; Huang et al. 2020) since a massive inflammatory cell infiltration and inflammatory cytokines secretion were found in patients' lungs, alveolar epithelial cells and capillary endothelial cells were damaged, causing acute lung injury. COVID-19 disease severity is associated to a cytokine profile resembling secondary haemophagocytic lymphohistiocytosis (sHLH), a hyperinflammatory syndrome commonly triggered by viral infections and characterised by a fulminant and fatal hypercytokinaemia with multiorgan failure. Cardinal features of sHLH include unremitting fever, cytopenias, and hyperferritinaemia; pulmonary involvement (including ARDS) occurs in approximately 50% of patients. Similar to sHLH, the cytokine profile of COVID-19 patients is characterized by increased interleukin (IL)-2, IL-7, granulocyte-colony stimulating factor, interferon-γ inducible protein 10, monocyte chemoattractant protein 1, macrophage inflammatory protein 1-α, and tumour necrosis factor-α. Predictors of fatality from a recent retrospective, multicentre study of 150 confirmed COVID-19 cases in Wuhan, China, included elevated ferritin (mean 1297.6 ng/ml in non-survivors vs 614.0 ng/ml in survivors; p<0.001) and IL-6 (p<0.0001), suggesting that mortality might be due to virally driven hyperinflammation. In hyperinflammation, immunosuppression is likely to be beneficial. However, corticosteroids are not routinely recommended and might exacerbate COVID-19-associated lung injury (Mehta et al. 2020). Therefore, there is a high need for using new treatment options with immunomodulatory and anti-inflammatory properties.

A study conducted on 452 patients with COVID-19 showed that severe cases tend to have high leukocytes counts and neutrophil-lymphocyte-ratio (NLR), low lymphocytes counts, as well as low percentages of monocytes, eosinophils, and basophils. The number of T cells significantly decreased, and more hampered in severe cases. Both, helper T cells and suppressor T cells in patients with COVID-19 were below normal levels, and lower level of helper T cells in severe group. The percentage of naive helper T cells increased, and memory helper T cells decreased in severe cases. Patients with COVID-19 also have lower level of regulatory T cells, and more obviously damaged in severe cases (Qin et al. 2020). Therefore, since lymphocytopenia is often seen in severe COVID-19 patients, the hypercytokinaemia caused by SARS-CoV-2 virus has to be mediated by leukocytes other than T cells (Shi et al. 2020).

Complications of COVID-19 patients include acute lung injury, shock, acute kidney injury, liver injury, gastrointestinal symptoms and acute respiratory distress syndrome (ARDS), which represents the leading cause of mortality (Singhal 2020; Rothan and Byrareddy 2020; Xu et al. 2020) and represent stage III of SARSCoV-2 infections.

Clinically, the immune responses induced by SARS-CoV-2 infection are two phased. During the incubation and non-severe stages, a specific adaptive immune response is required to eliminate the virus and to preclude disease progression to severe stages (Shi et al. 2020). However, when a protective immune response is impaired, virus will propagate and massive destruction of the affected tissues will occur, especially in organs that have high ACE2 expression, the virus entry receptor, such as lungs, arteries, heart, kidney, and intestines (Shi et al. 2020; Hamming et al. 2004). The damaged cells induce innate inflammation in the lungs that is largely mediated by proinflammatory macrophages and granulocytes. In addition, some of the cases show an infiltrate in the upper lobe of the lung that is associated with increasing dyspnea with hypoxemia (Rothan and Byrareddy 2020). Therefore, for a possible therapy the drug needs to fulfill three molecular characteristics: (1) anti-inflammatory function by interaction with macrophages, (2) immunomodulation by suppression of neutrophil granulocytes, and (3) hypoxia-induced secretion of VEGF to promote proliferation of epithelial cells, induced protection of vascular permeability, and prevented apoptosis of endothelial cells in the lungs. ABCB5-positive MSCs possess all of these properties (Vander Beken et al., 2019; Jiang et al., 2016).

In detail, in vitro and in vivo studies have shown the unique anti-inflammatory and immunomodulatory properties of ABCB5-positive cells in different animal models: in an ABCB5 knockout mouse model, a diabetic wound mouse model, a model with chronic wound in immunocompetent and humanized NSG mice, and acute wound mouse model. These studies demonstrated that ABCB5-positive cells trigger the switch from pro-inflammatory M1 macrophages (secreting pro-inflammatory cytokines TNF-α and IL-12/IL-23p40) to anti-inflammatory M2 macrophages (secreting anti-inflammatory cytokine IL-10) by secretion of IL-1RA. The receptor antagonist inhibits IL-1 signaling by binding to the IL-1 receptors without accessory protein docking. Thus, IL-1RA prevents downstream IL-1 signaling, promotes a M2 macrophage phenotype and anti-inflammation (Vander Beken et al. 2019). The secretion of IL-1RA is a reproducible and robust immunomodulatory capacity of the ABCB5-positive cells and thus defined as release criterion for the IMP: Every cell batch must prove their immunomodulatory potential by secretion of IL-1RA after co-cultivation with M1-polarized macrophages.

Furthermore, in a RDEB (Recessive dystrophic epidermolysis bullosa) mouse model from Tolar (Webber et al. 2017), intravenous administration of ABCB5-positive cells into neonate mice resulted in a markedly reduced RDEB pathology and a significantly extended lifespan. Tolar suspected an effect mechanism via reduced skin infiltration of inflammatory myeloid derivatives and modulation of macrophages, and thus suppression of inflammation.

Recently, it was shown that ABCB5 identifies programmed cell death 1 (PD-1) positive Immunoregulatory Dermal Cells (DIRCs) (Schatton et al. 2015). PD-1 is co-expressed with ABCB5 and these cells suppress T-cell proliferation and induce Tregs. Tregs inhibit proinflammatory properties of macrophages and can therefore suppress inflammation (Schatton et al. 2015), one of the key features of COVID-19.

Therefore, the results described above and provided in the Examples below indicate that ABCB5 cells modulate inflammation. The positive effects of the IMP can be attributed to increased anti-inflammatory mechanisms by secretion of anti-inflammatory cytokines such as IL-1RA and IL-10. The secretion leads to the suppression of pro-inflammatory cytokines like TNF-α and IL-1β, which mediate the necessary switch of macrophages from pro-inflammatory M1 to anti-inflammatory and pro-angiogenic M2 macrophages. Moreover, PD-1 is co-expressed with ABCB5 and further supports the anti-inflammatory and immunomodulatory properties of ABCB5-positive cells. Hypoxia-induced VEGF-secretion is confirmed for ABCB5-positive cells, which aligns with a phosphorylation of HIF la that is localized in the nucleus.

Without wishing to be bound by theory, it is thought that administration of ABCB5-positive cells (e.g., allo-APZ2-Covid19) will improve the clinical condition of patients suffering from inflammatory conditions, such as Covid19. The active substances of allo-APZ2-Covid19 are allogeneic ABCB5-positive cells from skin tissue that are expanded and isolated using a specific antibody.

Mesenchymal stem cells (MSCs) are known to migrate to damaged tissues, exert anti-inflammatory and immunoregulatory functions, promote the regeneration of damaged tissues and inhibit tissue fibrosis by interacting with the inflammatory microenvironment (Hoogduijn et al. 2010; Wada, Gronthos, and Bartold 2013; Wang et al. 2014).

ATP-binding cassette, sub-family B, member 5 (ABCB5)-positive skin progenitor cells reside in the reticular dermis and are distinct from neighboring mature fibroblasts, CD31⁺ endothelial cells, and bulge cells. Flow cytometric analyses of dissociated and propagated human skin specimens revealed ABCB5 to be expressed by 2.5-5% of all cells in healthy skin samples. ABCB5-positive cells co-expressed the MSC markers CD29, CD44, CD49e, CD90, and CD166, as well as the stem cell marker CD133, but were negative for differentiation markers such as the endothelial lineage marker CD31, the hematopoietic lineage marker CD45, and the quiescent fibroblast marker CD34. Importantly, only distinct subpopulations of cells staining positively for the reported MSC markers (CD29, CD44, CD49e, CD90 and CD166) stained positively for ABCB5, whereas large proportions of cells expressing these antigens were found to be negative for ABCB5, demonstrating that ABCB5-positive cells represent a unique novel subpopulation of MSC phenotype-expressing skin progenitor cells (Kim et al.

In some aspects the invention is a method of treating a subject having an inflammatory disorder, such as Covid19 with a composition comprising ABCB5-positive cells. In some embodiments, the composition comprises allo-APZ2-Covid19. The treatment, in some embodiments, is administered 1, 2, 3, 4, 5, 6, 7, 8, 9, 10, or more times. In some embodiments, the treatment is administered 3 times a day, twice a day, daily, every other day, every third day, every fourth day, every fifth day, every sixth day, weekly, biweekly, or monthly. In one embodiment, the treatment is administered every other day for three days (e.g., Day 0, Day 2, and Day 4). In some embodiments, the dose administered for each treatment is 1×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, 9×10⁷ cells, 1×10⁸ cells, 2×10⁸ cells, 3×10⁸ cells, 4×10⁸ cells, 5×10⁸ cells, 6×10⁸ cells, 7×10⁸ cells, 8×10⁸ cells, 9×10⁸ cells, 1×10⁹ cells, or more. In one embodiment, the dose administered for each treatment is 100×10⁶ cells. In some embodiments the concentration of cells administered is 1×10⁶ cells/mL, 1×10⁷ cells/mL, 2×10⁷ cells/mL, 3×10⁷ cells/mL, 4×10⁷ cells/mL, 5×10⁷ cells/mL, 6×10⁷ cells/mL, 7×10⁷ cells/mL, 8×10⁷ cells/mL, 9×10⁷ cells/mL, 1×10⁸ cells/mL, 2×10⁸ cells/mL, 3×10⁸ cells/mL, 4×10⁸ cells/mL, 5×10⁸ cells/mL, 6×10⁸ cells/mL, 7×10⁸ cells/mL, 8×10⁸ cells/mL, 9×10⁸ cells/mL, 1×10⁹ cells/mL, or more. In some embodiments, the concentration administered is 1×10⁷ cells/mL.

The treatment will usually be administered by intravenous injection or infusion (e.g., to a peripheral vein) although methods of implanting cells, e.g. near the site of infection, may be used as well.

ABCB5 is a novel and important marker for the isolation of multipotent stem cell populations from normal human tissue. “ABCB5(+) stem cells,” as used herein, refers to cells having the capacity to self-renew and to differentiate into mature cells of multiple adult cell lineages. These cells are characterized by the expression of ABCB5on the cell surface. In some embodiments of the invention, ABCB5(+) stem cells are dermal or ocular stem cells. In other embodiments the ABCB5(+) stem cells are synthetic stem cells.

“ABCB5positive dermal mesenchymal stem cells” as used herein refers to cells of the skin having the capacity to self-renew and to differentiate into mature cells of multiple adult cell lineages such as bone, fat and cartilage. These cells are characterized by the expression of ABCB5 on the cell surface. In culture, mesenchymal stem cells may be guided to differentiate into bone, fat, cartilage, or muscle cells using specific media. (Hirschi K K and, Goodell M A. Gene Ther. 2002; 9: 648-652. Pittenger M F, et al., Science. 1999; 284: 143-147. Schwartz R E, et al., J Clin Invest. 2002; 109: 1291-1302. Hirschi K and Goodell M. Differentiation. 2001; 68: 186-192.)

The ABCB5 positive dermal mesenchymal stem cells can be obtained from skin. The skin may be derived from any subject having skin, but in some embodiments is preferably human skin. The skin may be derived from a subject of any age but in some embodiments is preferably adult skin, rather than adolescent or infant skin.

ABCB5⁺ cells have been identified as a phenotypically distinct dermal cell population able to provide immunoregulatory functions. Greater than 90% of ABCB5⁺ cells express MSC markers CD29, CD44, CD49e, CD73, CD105, and CD166, as well as the immune checkpoint receptor PD-1.

In other embodiments of the invention, ABCB5(+) stem cells are ocular stem cells. ABCB5(+) stem cells may be obtained from (e.g., isolated from or derived from) the basal limbal epithelium of the eye or from the retinal pigment epithelium (RPE). In some embodiments, ABCB5(+) stem cells are obtained from human eye. Other ABCB5(+) stem cell types such as, for example, those obtained from the central cornea may be used in various aspects and embodiments of the invention.

The cells of the invention also may possess multipotent differentiation capacity. In other words these cells not only define mesenchymal stromal cells (adipogenic, chondrogenic, osteogenic differentiation), but also other capacities, including differentiation to cells derived from of all three germ layers, i.e. 1. endoderm (e.g. angiogenesis—e.g. tube formation, CD31 and VEGFR1 expression), 2. mesoderm (e.g. myogenesis—e.g. spectrin, desmin expression) and 3. ectoderm (e.g. neurogenesis—e.g. Tuj1 expression).

In other embodiments of the invention, ABCB5(+) stem cells are synthetic stem cells. ABCB5+ stem cells isolated from human tissue can be passaged in culture to produce populations of cells that are structurally and functionally distinct from the original primary cells isolated from the tissue. These cells are referred to herein as synthetic or manufactured ABCB5+ stem cells. These cells are in vitro manufactured such that nearly all cells are in vitro progeny of physiologically occurring skin-derived ABCB5-positive mesenchymal stem cells that never existed in the context of the human body. Rather, they are newly created. The compositions of the invention are populations of cells. The term “population of cells” as used herein refers to a composition comprising at least two, e.g., two or more, e.g., more than one, synthetic ABCB5+ stem cells, and does not denote any level of purity or the presence or absence of other cell types, unless otherwise specified. In an exemplary embodiment, the population is substantially free of other cell types. In some embodiments greater than 99%, 99.5%, 99.7%, 99.9%, 99.99%, 99.998%, 99.999%, or 99.999997% of the population is an in vitro progeny of physiologically occurring skin-derived ABCB5-positive mesenchymal stem cells.

The synthetic cells may also have distinct gene expression profiles relative to primary stem cells isolated from human tissue. The populations of synthetic cells (also referred to as ABCB5+ cells isolated from high passages) are different from the primary cells (those derived from low passage cultures that contain the native ABCB5+ cells found in the living organism). For example, certain stem cell markers are increased in high passage cells, e.g. SOX2, NANOG and SOX3, while certain mesenchymal stromal differentiation markers are decreased, e.g. MCAM, CRIG1 and ATXN1. The expression of selected stemness markers such as SSEA-4, DPP4 (CD26), PRDM1 (BLIMP1) and POU5F1 (OCT-4) in ABCB5+ cells in human skin at protein level was confirmed by immunostaining. While the expression of lower fibroblast lineage marker α-smooth muscle actin (α-SMA) was absent in ABCB5+ cells of human skin. These data support the finding that these late passage synthetic cells maintain pluripotent properties of ABCB5+ cells, and even have enhanced properties relative to the original cells.

In some preferred embodiments, 100% of the cells are synthetic, with 0% of the cells originating from the human tissue.

The ABCB5+ stem cells used herein are preferably isolated. An “isolated ABCB5+ stem cell” as used herein refers to a preparation of cells that are placed into conditions other than their natural environment. The term “isolated” does not preclude the later use of these cells thereafter in combinations or mixtures with other cells or in an in vivo environment.

The ABCB5+ stem cells may be prepared as substantially pure preparations. The term “substantially pure” means that a preparation is substantially free of cells other than ABCB5 positive stem cells. For example, the ABCB5 cells should constitute at least 70 percent of the total cells present with greater percentages, e.g., at least 85, 90, 95 or 99 percent, being preferred. The cells may be packaged in a finished pharmaceutical container such as an injection vial, ampoule, or infusion bag along with any other components that may be desired, e.g., agents for preserving cells, or reducing bacterial growth. The composition should be in unit dosage form.

In the embodiments when the ABCB5+stem cells are administered to a subject the cells may be autologous to the host (obtained from the same host) or non-autologous such as cells that are allogeneic or syngeneic to the host. Non-autologous cells are derived from someone other than the patient. Alternatively the ABCB5+stem cells can be obtained from a source that is xenogeneic to the host.

Allogeneic refers to cells that are genetically different although belonging to or obtained from the same species as the host or donor. Thus, an allogeneic human mesenchymal stem cell is a mesenchymal stem cell obtained from a human other than the intended recipient of the ABCB5+stem cells. Syngeneic refers to cells that are genetically identical or closely related and immunologically compatible to the host or donor, i.e., from individuals or tissues that have identical genotypes. Xenogeneic refers to cells derived or obtained from an organism of a different species than the host or donor.

When cells are administered an effective dose of cells should be given to a patient. The number of cells administered should generally be in the range of 1×10⁷ -1×10¹⁰ and, in most cases should be between 1×10⁸ and 5×10⁹, or more specifically one of the doses discussed above. Actual dosages and dosing schedules will be determined on a case by case basis by the attending physician using methods that are standard in the art of clinical medicine and taking into account factors such as the patient's age, weight, and physical condition. The cells will usually be administered by intravenous injection or infusion although methods of implanting cells may be used as well.

The ABCB5+stem cells may be modified to express additional proteins which are also useful in the therapeutic indications, as described in more detail below. For example, the cells may include a nucleic acid that produces at least one bioactive factor which enhances ABCB5+stem cell activity. Thus, the ABCB5+stem cells may be genetically engineered (or transduced or transfected) with a gene of interest. Thus, the ABCB5+ stem cells, and progeny thereof, can be genetically altered. Genetic alteration of an ABCB5+ stem cell includes all transient and stable changes of the cellular genetic material which are created by the addition of exogenous genetic material. Exogenous genetic material includes nucleic acids or oligonucleotides, either natural or synthetic, that are introduced into the ABCB5+stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half-life, and the like. Polymeric delivery systems also have been used successfully to deliver nucleic acids into cells, as is known by those skilled in the art. Such systems even permit oral delivery of nucleic acids.

One method of introducing exogenous genetic material into the ABCB5+stem cells is by transducing the cells using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

A major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of ABCB5+stem cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target mesenchymal stem cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into dermal synthetic ABCB5+stem cells. The selection of an appropriate vector to deliver a therapeutic agent for a particular condition amenable to gene replacement therapy and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the dermal mesenchymal stem cell genome immediately downstream from the promoter so that the promoter and coding sequence are operatively linked so as to permit transcription of the coding sequence. A preferred expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified dermal mesenchymal stem cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the subject.

In addition to at least one promoter and at least one heterologous nucleic acid encoding the therapeutic agent, the expression vector preferably includes a selection gene, for example, a neomycin resistance gene, for facilitating selection of ABCB5+stem cells that have been transfected or transduced with the expression vector. Alternatively, the ABCB5+stem cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

The selection and optimization of a particular expression vector for expressing a specific gene product in an isolated stem cell is accomplished by obtaining the gene, preferably with one or more appropriate control regions (e.g., promoter, insertion sequence); preparing a vector construct comprising the vector into which is inserted the gene; transfecting or transducing cultured dermal synthetic ABCB5+stem cells in vitro with the vector construct; and determining whether the gene product is present in the cultured cells.

Thus, it is possible to genetically engineer ABCB5+stem cells in such a manner that they produce polypeptides, hormones and proteins not normally produced in human stem cells in biologically significant amounts or produced in small amounts but in situations in which overproduction would lead to a therapeutic benefit.

In some aspects, the disclosure provides for a method of treating hyper-inflammatory disorders. Hyperinflamatory diseases are diseases associated with excessive cytokine production or activation such as Interleukin-1. Examples of hyper-inflammatory or auto-inflammatory disorders include hereditary periodic fever syndromes (FMF), HIDS, TRAPS, FCAS, MWS, CINCA/NOMID), granulomatous inflammation (Crohn's disease, Blau syndrome, early onset sarcoidosis), complement disorders (Hereditary angioedema), pyogenic disorders (PAPA, CRMO), and vasculitis syndromes (Behcet's disease).

Recent identification of the molecular causes for Hereditary Periodic Fever Syndromes has led to improved understanding of their underlying cell biology and enabled targeted therapies for these diseases. Familial Mediterranean Fever (FMF) is caused by mutations in the MEFV gene. The MEFV gene encodes for pyrin protein, and is expressed mainly in neutrophils and monocytes. Pyrin is involved in the interleukin 1 inflammatory pathway and defective pyrin may lead to augmented inflammation through increased T-helper 1 activity. Disease severity varies according to the mutation present, and M694V is associated with a more severe phenotype. Development of amyloidosis leading to renal failure is the most important complication of FMF.

Hyperimmunoglobulin D with Periodic Fever Syndrome (HIDS) is caused by mutations in the mevalonate kinase gene (MVK). Mevalonate kinase is a key enzyme in the cholesterol metabolic pathway, and the activity of the enzyme is reduced to 5-10% of normal in HIDS. TNF Receptor-associated Periodic Syndrome (TRAPS) is caused by mutations in the TNF receptor 1 (TNFR1) gene, TNFR1A. TNRF1 is normally shed from receptors on cell surfaces, producing a pool of potentially TNF-neutralizing soluble TNRF1 in the plasma. Most inflammatory attacks are a consequence of a defect in the shedding of TNRF1, leading to increased cell surface expression and reduced circulating TNRF1. Familial Cold Auto-inflammatory Syndrome (FCAS), Muckle-Wells syndrome (MWS), and Chronic Infantile Neurologic, Cutaneous and Articular Syndrome/Neonatal-onset Multi-systemic Inflammatory Disease (CINCA/NOMID) are caused by mutations in the CIAS1 gene encoding cryopyrin. They were once considered three distinct diseases, but actually represent a continuum of clinical severity, with FCAS being the mildest, MWS being intermediate and CINCA/NOMID having the most severe disease. The majority of mutations cluster within a highly conserved NACHT domain resulting in spontaneous caspase-1 activation and excessive interleukin-1β production.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

As noted herein, mesenchymal stem cells are known for their unique immunomodulatory and anti-inflammatory effects (Baraniak and McDevitt 2010), which underlines the potential role in the treatment of a disease like COVID-19. COVID-19 is characterized by severe systemic inflammation which leads to organ failures and finally death.

Allo-APZ2-Covid19 falls into the scope of the Committee for Medicinal Products for Human Use's (CHMP) Guidelines and is classified as an advanced therapy medicinal product (ATMP). The nonclinical testing strategy described below relies on recommendations outlined in the CHMP Guidelines.

Accordingly, special emphasis was put on the product characterization and comparability between the cell-based products used in non-clinical and the planned clinical Phase I/IIa study in patients. All ABCB5-positive MSCs used in the following studies were isolated by antibody-coupled magnetic beads from explant cultures of different human donors and stored in the gas-phase of liquid nitrogen (cryo-preserved using CryoStor CS10 Freeze Medium containing 10% DMSO). Further, all of the batches of ABCB5-positive cells used were produced under GMP-conditions in the clean rooms and released after completing defined release criteria. ABCB5-positive cells were thawed, washed, and suspended in HRG solution for use in the studies described below.

As described below, preliminary in vitro and in vivo mode-of-action studies confirmed that treatment with ABCB5-positive cells is beneficial in modulating of inflammatory processes. The underlying mechanisms included the rebalancing of unrestrained cytokine levels, e.g. secretion of IL-1RA and IL-10 leading to TNF-α and IL-1β suppression, triggering the important macrophage switch towards anti-inflammatory and pro-angiogenic M2 macrophages and increased angiogenesis.

### Example 1

### Pharmacodynamics

First, the location of ABCB5-positive stromal cells was examined. Human and murine dermis were found to harbor ABCB5-positive stromal cells in the perivascular and interfollicular niche.

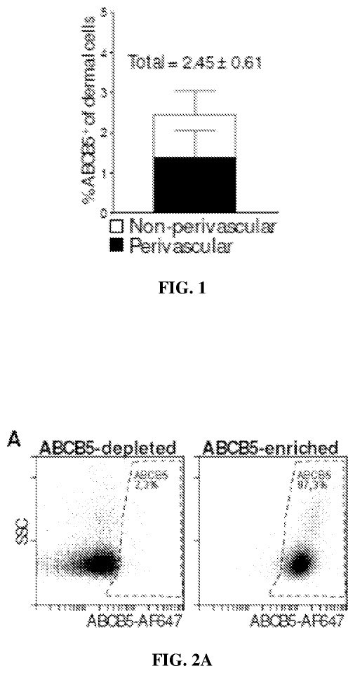

Using immunostaining of healthy human skin sections, ABCB5-positive cells were found to be either confined to a perivascular endogenous niche, in close association with CD31⁺ endothelial cells or dispersed within the interfollicular dermis independent of hair follicles (FIG. 1). ABCB5-positive cells constituted 2.45% ±0.61% of all dermal cells in the skin of ten different donors and of the ABCB5-positive cells, 55.3% ±23.9% were localized perivascularly, which was defined as a maximum of one additional cell in between the CD31⁺ endothelial cell and the ABCB5-positive cells (FIG. 1). Perivascular ABCB5-positive cells were clearly distinct from neural/glial antigen 2 (NG2) positive pericytes, as these markers did not co-localize in double immunostained human skin sections. In addition, dermal ABCB5-positive cells stained positive for the carbohydrate stage-specific embryonic antigen-4 (SSEA-4), an embryonic germ and stem cell marker earlier reported to be expressed on MSCs in different adult tissues, including the dermis. A similar distribution of ABCB5-positive cells in their endogenous niche was found in murine skin.

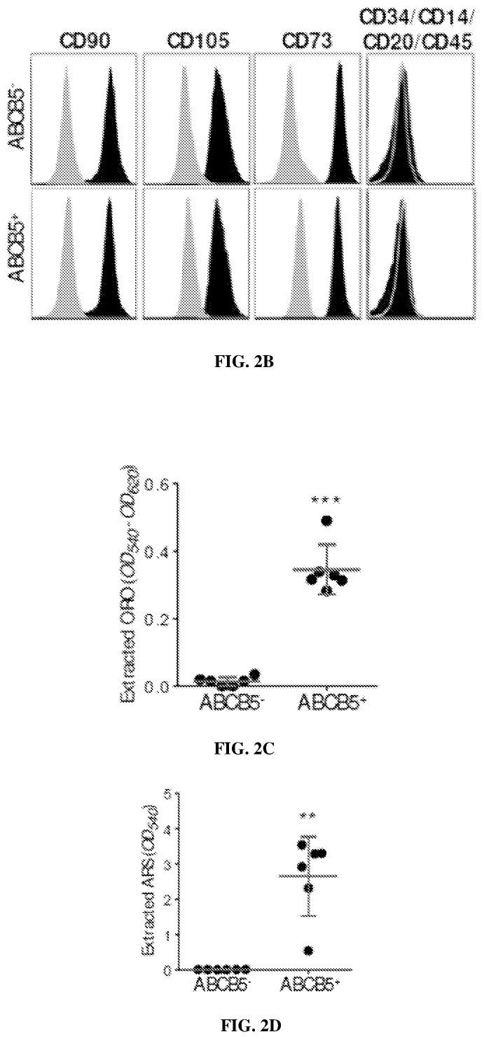

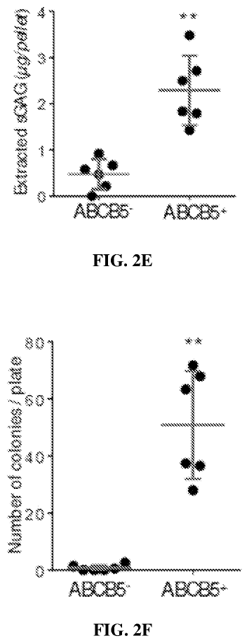

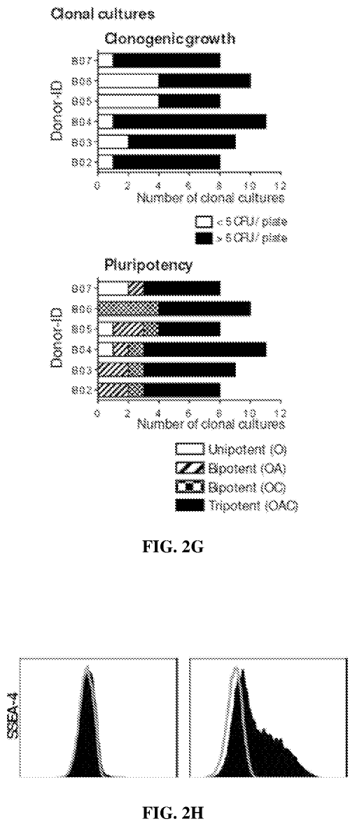

Human dermal ABCB5 cells are enriched for mesenchymal stem cells. To assess whether selection for ABCB5 in vitro results in a cell fraction enriched for MSCs, dermal single cell suspensions derived from enzymatically digested skin were plated on plastic tissue culture plates and following expansion (at the maximum for 16 passages equaling a cumulative population doubling of 25), the plastic adherent fraction was separated by multiple rounds of ABCB5 magnetic bead sorting. This resulted in two different cell fractions, a double ABCB5-enriched fraction containing on average 98.33% ±1.12% ABCB5-positive cells and a threefold ABCB5-depleted fraction, that only contained a very low percentage of ABCB5-positive cells as illustrated with flow cytometry dot plots for cells from donor B01 (FIG. 2A). Both ABCB5-positive and ABCB5-negative fractions displayed a fibroblastoid, spindle-like cell morphology (not shown) and expressed the characteristic minimal set of mesenchymal lineage markers CD90, CD73 and CD105, while no expression of hematopoietic stem cell and lineage markers CD14, CD20, CD34 and CD45 was detected by flow cytometry (FIG. 2B). A consistent and significantly increased potential for adipogenic, osteogenic and chondrogenic lineage differentiation was observed for ABCB5-positive cells as compared to donor-matched ABCB5-depleted cells (FIGS. 2C-2E), thereby delineating the ABCB5-positive cell fraction as multipotent adult MSCs from ABCB5-negative human dermal fibroblasts (HDFs). This was further confirmed by the finding that ABCB5 magnetic bead-sorted cells gave rise to single cell derived colonies, whereas the ABCB5-depleted fractions did not (FIG. 2F). To assess the in vitro self-renewal capacity of ABCB5-positive cells, subclonogenic growth and trilineage differentiation potential of 54 clonal cultures of ABCB5 sorted MSCs from six different donors were determined (FIG. 2G). It was found that 75.61 ±16.86% of clonal colonies again displayed clonogenic growth and 62.40 ±7.54% of all studied clones, generated from a single cell, and maintained their potential to differentiate into all three mesenchymal cell lineages. An additional 29.84 ±11.57% of these clones were bipotent, and 7.77 ±10.02% were unipotent for osteogenic differentiation. None of the clones from six donors were negative for all three lineages. In contrast to triple ABCB5-depleted cells, the ABCB5-positive sorted cell fractions revealed distinct stem cell associated SSEA-4 (Vaculik et al. 2012) expression (FIG. 2H). Nuclei of ABCB5-positive cells grown on 149 slides stained positive for SOX2, the stem cell-associated transcription factor sex determining region Y-box 2, whereas ABCB5-negative cells did not (FIG. 2H). Neither ABCB5-positive nor ABCB5-negative dermal plastic-adherent cell fractions expressed the additionally tested cell surface markers Melan-A (melanocytic cells), CD318 (epithelial cells) and CD271 (a neurotrophic factor found on other MSC populations).

Anti-Inflammatory Effects of ABCB5-positive Cells

ABCB5-positive cells were co-cultured with allogeneic PBMC CD14 ⁺ monocyte-derived macrophages that had been activated with recombinant human IFN-γ and LPS. Of note, significantly less M1 macrophage derived pro-inflammatory cytokines TNF-α and IL-12/IL-23p40 were detected in supernatants when activated macrophages were co-cultured with ABCB5-positive cells, as opposed to co-cultures with donor-matched ABCB5-negative Fibroblasts or macrophages cultured alone. Conversely, increased amounts of the M2 macrophage derived anti-inflammatory cytokine IL-10 were found in supernatants of macrophages co-cultured with ABCB5-positive cells compared to donor-matched ABCB5-negative HDFs or macrophages cultured alone.

To obtain further insights into mechanisms underlying the anti-inflammatory effects of ABCB5-positive cells in vivo, ABCB5-positive and ABCB5-negative cells were injected intradermally (i.d.) around the wound edges at day one after wounding in iron overload non-immunosuppressed mice. Inflammation was addressed by measuring cytokine expression in total protein lysates of day 5 wounds by enzyme-linked immunosorbent assay. Highly increased titers of TNF-α (M1-marker) and IL-1β (M1-marker) were measured in chronic wounds from iron-treated mice as compared to the dextran-treated acute control wounds. Injection of ABCB5-positive cells, but not of the donor-matched ABCB5-negative dermal cells, could significantly counteract this pro-inflammatory cytokine profile and additionally mediated a marked increase in production of the anti-inflammatory cytokine IL-10 (M2-marker) in chronic murine wounds.

Furthermore, NSG mice, humanized with PBMC, were used to validate the effect of ABCB5-positive cell injection on the M1/M2 wound macrophage phenotype of human origin in NSG iron overload mice. Co-immunostaining of day five wounds with human specific anti-CD68 and either anti-CD206 or anti-TNFα showed a higher number of CD68⁺ CD206⁺ human M2 macrophages in the wound beds of ABCB5-positive cells-injected compared to PBS-injected wounds, while the number of CD68⁺ TNFα⁺ pro-inflammatory macrophages was decreased in ABCB5-positive cell compared to PBS-injected wounds.

Schatton et al. published that ABCB5 identifies PD-1 positive immunoregulatory dermal cells (Schatton et al. 2015). PD-1 is co-expressed with ABCB5, and ABCB5-positive/PD-1-positive cells suppress T-cell proliferation and induce T_{regs}. T_{regs} inhibit proinflammatory properties of macrophages and can therefore suppress inflammation, one of the key features of ACLF.

To confirm the anti-inflammatory properties of ABCB5-positive MSCs after systemic administration, a liver diseases model was used that is characterized by massive inflammation (Hartwig et al. 2019). In a collaboration with Prof. Steven Dooley (Universitasklinikum Mannheim) the influence of the IMP was investigated in the Mdr2-knockout mouse model. On a molecular level, inflammatory markers and fibrosis markers were investigated. There was a significant reduction of Colla in non-immunosuppressed transgenic animals and first signs of changes in levels of pro-inflammatory cytokines, e.g. TNFα, confirming the anti-inflammatory mechanism. Summarizing, preclinical data obtained in a transgenic mouse model with liver inflammation and damage shows beneficial effects of treatment with the IMP after i.v. administration. A possible mechanism for the anti-fibrotic properties is the secretion of anti-inflammatory molecules that cause inhibition of stellate cells of the liver. Stellate cells are known to be activated in liver fibrosis and to mediate collagen production. The more pronounced effect in mice without immunosuppression is a good indicator for the importance of the immunomodulatory properties of the ABCB5-positive cells. The immunomodulatory and anti-inflammatory effects are expected to support the resolution of the systemic inflammation of COVID-19 patients.

Furthermore, the immunomodulatory function of ABCB5 was confirmed after systemic administration using an NSG RDEB KO mice model (Webber et al. 2017b). Bi-allelic knockout animals exhibited severe blisters within 24 hours of birth which lead to death of these animals within the first 2 weeks of their life. When these animals were transplanted with ABCB5+ MSCs, they showed marked improvement regarding blistering and survival. Long-term surviving of treated animals had a scruffier appearance of their coat compared to their wild-type littermates and even had evidence of pseudosyndactyly, however, they were generally in good health. Interestingly, none of the long-term surviving animals was positive for type VII collagen in this NSG model either by immunofluorescence microscopy or by quantitative PCR for human DNA.

Without wishing to be bound by theory, it is thought that the positive effect seen on the RDEB animals was due to an amelioration of their inflammatory condition. Tolar et al. hypothesized that this was a result of an effect of a mechanism of the ABCB5⁺ MSCs by suppression of early monocyte-mediated inflammation. The group investigated their hypothesis by assessing dermis infiltration of CD68+ macrophages in the damaged skin of RDEB mice. They observed a significant drop in CD68⁺ macrophages as soon as 48 hours post-ABCB5⁺ MSC injection compared to the control group. In conclusion, ABCB5+ MSCs mediate their effects by a strong suppression on early inflammation macrophages. This interaction was sufficient to rescue the RDEB phenotype and to allow the knockout mice to survive past crisis.

The mechanism of action for allo-APZ2-Covid19 does not predict an effect on non-target physiological systems. The present toxicity package does not point to any secondary pharmacodynamic effects. No secondary pharmacodynamics studies were performed.

No pharmacology studies were performed. The lack of safety pharmacology studies is considered justified as it is not anticipated that a cellular product of the nature of allo-APZ2-Covid19 will induce effects on vital functions (central nervous system, cardiovascular, respiratory) after systemic administration.

### Example 2

### Pharmacokinetics and Biodistribution

A biodistribution and persistence study after a single intravenous (i.v.) dose was performed in NOD-SCID mice and NOD-SCID gamma (NSG) mice, respectively to investigate trafficking, homing, engraftment, differentiation, and persistence of ABCB5-positive cells in target and non-target body tissues following a single i.v. injection to male and female NOD/SCID/IL2Rγ^{null} (NSG) mice followed by a 1-13 week observation period. Vehicle (HRG; HSA, ringer lactate, and glucose) or 2×10⁶ ABCB5-positive cells in vehicle were administered to NOD/SCID/IL2Rγ^{null} (NSG) mice (n=5/sex/group), age 7-8 weeks, by a single i.v. injection into the left or right caudal veins with a 26G needle at a volume of 200 μl. The groups are shown in the Table 1 below.

TABLE 1
Experimental Groups and Doses

| | | | | | ABCB5- | | |
|---|---|---|---|---|---|---|---|
| | Number | | | | positive MSC | | |
| | of | | | Observation | Suspension | Admin. | Dose Level |
| Group | Animals | Treatment | Route | Period | [cells/ml] | Volume | [cells/animal] |
| 1 | 5 M | Vehicle | i.v. | 13 weeks | — | 200 μl | — |
| | 5 F | | | | | | |
| 2 | 5 M | ABCB5- | i.v. | 1 week | 1 × 10⁷ | 200 μl | 2 × 10⁶ |
| | 5 F | positive | | | | | |
| | | cells | | | | | |
| 3 | 5 M | ABCB5- | i.v. | 4 weeks | 1 × 10⁷ | 200 μl | 2 × 10⁶ |
| | 5 F | positive | | | | | |
| | | cells | | | | | |
| 4 | 5 M | ABCB5- | i.v. | 13 weeks | 1 × 10⁷ | 200 μl | 2 × 10⁶ |
| | 5 F | positive | | | | | |
| | | cells | | | | | |

Blood and tissue samples were collected at pre-determined time points up to 13 weeks after treatment and analysed for the determination of distribution across tissues.

A variety of parameters as mortality, daily cage side observations, weekly detailed clinical observations and body weight were determined. Animals were necropsied after 1 week (Day 8), 4 weeks (Day 29) and 13 weeks (Day 92). For PCR analysis, several tissues were collected. Organ sampling for qPCR includes skin/subcutis (injection site; tail section), skeletal muscle (injection site; tail section) and lymph nodes near injection site, liver, spleen, lung, brain, femur bone with bone marrow, kidney, thymus, thyroid/parathyroid gland, ovaries/testes, blood.

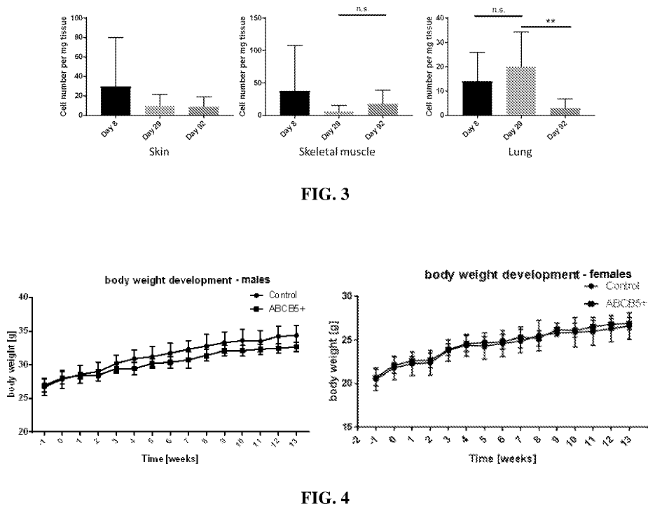

The detection of the test item in the different tissues was performed by semi-quantitative detection of human-specific DNA-sequences via TaqMan-PCR (qPCR). The quality and amount of the total DNA was monitored by applying a TaqMan-PCR detecting a mouse-specific DNA-sequence. PCR analysis was performed under GLP conditions.
Mortality and Clinical Signs: There were no deaths during the study as a consequence of reaction to treatment. Bruising around the injection site was observed in the majority of animals across sexes and groups, however there was no clear difference in incidence between treated and vehicle control groups and as such this is considered consequential to the route of administration. Therefore, no clinical findings considered related to treatment were observed during the study.
Body Weight: Group mean body weight gain was less than controls for Group 4 males in Week 9 and for Group 4 females during Weeks 4-6 and 10-12; however, this was considered a consequence of individual variation and not indicative of any treatment related effect.
Biodistribution/PCR Analysis: In the course of the study, 10 PCR assays were performed. All assays met the acceptance criteria and were declared as valid. Recovery of the tissue control samples (TQCs) was comparable for all extractions. Biodistribution analysis revealed DNA from target MSCs present above the limit of quantification (>125 cells) in 11% of the tissue samples analysed up to 92 days post dose. Findings for different tissues were as follows:
   Injection site tissues: For treated animals, MSCs were predominantly determined at the injection site (skin and skeletal muscle) and were detectable in individual animals up to Day 92 in both sexes (Table 2). No consistent sex difference was observed. Positive results were obtained in 50-60% (skin) and, respectively, 30-50% (skeletal muscle) of all animals per group throughout the study. ABCB5-positive cell concentrations appeared to be at maximum on Day 8 (up to 162 and, respectively, 200 cells/mg), generally declining in concentration thereafter in these tissues: On day 92, maximal 31 cells (skin) and 57 cells (skeletal muscle) per mg tissue were found (note that the apparent increase on day 92 compared to day 29 for skeletal muscle tissue was found to be non-significant). Hence, applied cells seem to stay at the injection site for a limited period of time and vanish afterwards — as expected in NSG mice.
   Lung: Detection of administered ABCB5-positive cells in lung tissue after intravenous application is well known, due to its filtering effect on passing cells (Kean et al. 2013). In accordance with this, ABCB5-positive MSCs were found at low to medium concentrations (up to 44 cells per mg) at this location in both, male and female mice (Table 2). Just like in injection site tissues, a time-dependent decrease of human cells was shown (FIG. 3). Attention should be paid to the significant reduction between day 29 and day 92. In contrast to this, illusive proliferation during the first month was found to be non-significant, supporting the assumption of initial persistence but long-term degradation.
   Kidney, liver, thymus and femur bone: No positive outcomes were found for all animals in kidney, liver, thymus and femur bone (with bone marrow) tissue. Merely, one of thirty treated animals (male, #13, day 29) depicted a slightly positive finding (7 cells/mg) for kidney and liver. Re-analysis of the DNA-eluate samples resulted in a signal below the lower limit of quantification (termed as “detected”) for both tissues. Additional DNA re-extraction from residual tissue depicted 15 cells/mg in the kidney sample whereas liver remained unquantifiable. Notably, no positive findings were obtained in these tissue types at previous time points. Concluding this marginal and only partial reproducible outcome, these findings are assessed as incidental and especially regarding the kind of tissues evaluated as non-safety relevant.
   In addition to this, high numbers of human cells (151 cells/mg) were found in thymus tissue of male #11 on day 29. Apart from this mouse, all other rodents were found inconspicuous regarding thymus. Re-examination of the DNA-eluate confirmed the presence of human cells (193 cells/mg) and the reliability of the qPCR. Due to limited amounts of homogenate, further investigations (DNA re-extraction) were not possible. Since, this animal was the only one which showed positive signals in this tissue type (in particular concerning samples from previous time points), it is assumed to be either incidental or related to contamination at an undetermined stage of the study. Even if this finding represents a true positive, contamination-unrelated result it would not be evaluated as safety-relevant, because a combined toxicity/tumorigenicity study (study no. FN9OBS) with i.v. administered ABCB5-positive MSCs did not show any test item-related effect (in particular not in the thymus).

Finally, human cells were observed at low levels (7 cells/mg) in the femur bone of female #38 (day 8). DNA-eluate re-analysis confirmed this result (7 cells/mg); however, re-testing of tissue leftover refuted it by finding no human cells. No other animal depicted quantifiable numbers of human cells for this tissue. Based on these data, this finding is considered to be unreliable as well and non-relevant regarding safety.
Brain, lymph nodes, spleen, thyroids, testes/ovaries and blood: No quantifiable numbers of human cells were found in the residual tissues and organs in all animals. Especially brain tissue and reproductive organs demonstrated absence of MSCs. Therefore, these outcomes demonstrate safety of ABCB5-positive MSCs.

TABLE 2
Numbers of human cells in test item-treated animals, detected
by qPCR in tissues of the injection site and downstream thereof

| | | | Cell Concentration (cell/mg | | |
|---|---|---|---|---|---|
| | | | tissue) | | |
| | | | Injection site | | |
| | | | tissues | | |
| | | | | Skeletal | |
| Group | ex | Animal # | Skin | muscle | Lung |

| | | | | | |
|---|---|---|---|---|---|
| Treated with | Day 8 | 6 | 8 | − | 36 |
| ABCB5+ MSCs | | 7 | 162 | detected | − |
| | | 8 | 59 | 46 | 14 |
| | | 9 | 13 | − | 11 |
| | | 10 | − | detected | − |
| | | 36 | detected | 200 | 12 |
| | | 37 | detected | 129 | 7 |
| | | 38 | 16 | detected | 31 |
| | | 39 | 39 | − | 19 |
| | | 50 | detected | − | 10 |
| | Day 29 | 11 | − | − | 15 |
| | | 12 | 21 | detected | 15 |
| | | 13 | 18 | − | 12 |
| | | 14 | detected | detected | 30 |
| | | 15 | 7 | detected | 10 |
| | | 40 | − | − | − |
| | | 41 | 33 | 19 | 44 |
| | | 42 | 18 | 24 | 7 |
| | | 43 | detected | 15 | 35 |
| | | 51 | − | − | 32 |
| | Day 92 | 16 | 17 | − | − |
| | | 17 | − | 57 | 7 |
| | | 18 | − | − | 6 |
| | | 19 | 31 | 46 | − |
| | | 20 | 11 | 35 | 9 |
| | | 45 | − | − | − |
| | | 46 | detected | − | − |
| | | 47 | 8 | − | − |
| | | 48 | 13 | 17 | 7 |
| | | 49 | 8 | 18 | − |

− = not detected
n.a. = not applicable
detected = values <Lower limit of quantification but >2.5x Blank.

Single intravenous administration of human ABCB5-positive MSCs was well tolerated with no clinical findings at 2×10⁶ cells/animal. Investigation of the tissue distribution showed that quantifiable levels were generally confined to the lungs and injection site tissues (skin and skeletal muscle). Up to the end of the study on Day 92, detectable levels of DNA were persistent at the injection site and lungs, slightly increasing on Day 8 for injection site tissues and Day 29 for lung tissues followed by a significant reduction on Day 92. An additional study in order to evaluate the status of the remaining cells is conducted.

### Immunohistochemical Analysis

The purpose of the study was to evaluate the presence and proliferation status of remaining ABCB5-positive mesenchymal stem cells in lungs and injection sites. The frozen lung tissues of all animals examined used in above were available for histological investigation. Complete skin tissue sets were available from Group 2 and 3 of study BW35YB, but only of five animals of Group 4 (2 males [No. 16, 19], 3 females [No. 46, 48, 49]). Skin samples of the other five animals [No. 17, 18, 20, 45, 47] were used up for the qPCR analysis and thus no skin tissue of these animals could be investigated.

Frozen tissues were thawed briefly at room temperature, fixed in 10% Neutral Buffered Formalin (NBF) for 24-48 hours, then processed through to paraffin wax using an automated tissue processor. The processed tissues were then embedded into paraffin wax blocks.

Tissues were sectioned at three levels approximately 100 p.m apart. At each level three sequential sections 4-5 p.m in thickness were taken, one for Haematoxylin and Eosin (H&E) staining, to aid histopathological examination, one for immunohistochemistry using an Anti-mitochondrial antibody (AMA). If the AMA-staining was positive (i.e. detected human cells), the corresponding third slide was then stained with Ki67 antibody.

During the reporting phase, one sample was identified as positive for both AMA and Ki67. The study was therefore extended to investigate co-localization of these antigens. In Table 3 material used and corresponding groups of study are listed.

TABLE 3
Animal tissues used for immunostaining and corresponding groups

| | | | | Histological analysis (XP90QD) | |
|---|---|---|---|---|---|
| | Number of | Treatment | Observation | Lung tissues | Skin tissues |
| Group | Animals in | in | Period in | from (animal | from (animal |
| No. | BW35YB | BW35YB | BW35YB | no.) | no.) |
| 1 | 5 M | Vehicle | 13 weeks | 2 M (4, 5) | — |
| | 5 F | | | 2 F (30, 32) | — |
| 2 | 5 M | ABCB5- | 1 week | 5 M (6-10) | 5 M (6-10) |
| | 5 F | positive | | 5 F (36-39, 50) | 5 F (36-39, 50) |
| | | cells | | | |
| 3 | 5 M | ABCB5- | 4 weeks | 5 M (11-15) | 5 M (11-15) |
| | 5 F | positive | | 5 F (40-43, 51) | 5 F (40-43, 51) |
| | | cells | | | |
| 4 | 5 M | ABCB5- | 13 weeks | 5 M (16-20) | 2 M (16, 19) * |
| | 5 F | positive | | 5 F (45-49) | 3 F (46, 48, 49) * |
| | | cells | | | |

* No remaining skin tissue available for animals M17, M18, M20, F45, F47 (all Group 4 in BW35YB)

Staining with Anti-mitochondrial antibody (AMA): Positive staining for AMA was seen in samples from only 2 animals, one time at the injection site and for the other one in the lungs. Both were in group 2 during the study, which means that they were sampled one week after cell application.

Skin samples of 25 animals and tissues of 24 mice were investigated, and no positive staining was seen. At the injection site (skin/muscle) a single animal (2F 38) showed positive staining. The staining was seen in a focal cluster of spindloid cells, the appearance of which was consistent with cells of mesenchymal origin.

With respect to lung tissue, 30 animals were investigated of which 29 were negative. Positive staining for AMA was seen focally within a blood vessel of a single animal (2F 36). This staining appeared to be within a thrombus in the blood vessel. All positively staining cells were spindloid and therefore their morphology was consistent with cells of mesenchymal origin. All cells were arranged haphazardly throughout the thrombus and did not show any evidence of clustering together to form a mass.
Staining with Ki67 antibody: At the injection site of animal 2F 38, no positive staining for Ki67 was observed. The areas that showed positive staining for AMA were clearly identified on the sections stained for Ki67 and were negative.

In the lungs of animal 2F 38 positive staining for Ki67 was observed within the same thrombus that showed positive staining for AMA. The majority of cells that stained positively for Ki67 within the thrombus were plump cells with oval shaped nuclei and were therefore morphologically very different from the cells that stained positively for AMA. However, several cells that stained positively for Ki67 were spindloid in shape. Positive staining (for Ki67) cells of host origin and of varying morphologies would be expected within a thrombus due to active reorganization of the thrombus. However, it cannot be excluded for this one animal that some of the cells that stained positively for AMA also stained positively for Ki67 and were therefore both of human origin and actively proliferating.

Results of the single immunostaining experiments are shown in Table 4 below.
Dual Ki67 and AMA Staining: Dual staining was undertaken to determine whether any cells within the previously identified thrombus of the lung of animal 2F 36 were of human origin and actively proliferating. Positive staining for both Ki67 and AMA was seen in several cells within the thrombus, however staining for Ki67 did not appear to be specific in that particular run. Further optimization of the method was performed but unfortunately the region of interest had been exhausted by this time due to repeated sectioning. Consequently, it cannot be confidently excluded that some of the cells that stained positively for AMA also stained positively for Ki67 and were therefore both of human origin and actively proliferating.

TABLE 4
Results of immunostaining of tissues

| Group | Sample ID | AMA Staining | Ki67 Staining |
|---|---|---|---|
| Vehicle - | 1M 4 - Lung | − | N/A |
| 13 weeks | 1M 5 - Lung | − | N/A |
| | 1M 30 - Lung | − | N/A |
| | 1F 32 - Lung | − | N/A |
| ABCB5 - | 2M 6 - Lung | − * | N/A |
| positive | 2M 6 - Injection site | − | N/A |
| cells - | 2M 7 - Lung | − | N/A |
| 1 week | 2M 7 - Injection site | − * | N/A |
| | 2M 8 - Lung | − | N/A |
| | 2M 8 - Injection site | − * | N/A |
| | 2M 9 - Lung | − | N/A |
| | 2M 9 - Injection site | − | N/A |
| | 2M 10 - Lung | − | N/A |
| | 2M 10 - Injection site | − | N/A |
| | 2F 36 - Lung | ++ | ++ |
| | 2F 36 - Injection site | − | N/A |
| | 2F 37 - Lung | − | N/A |
| | 2F 37 - Injection site | − | N/A |
| | 2F 38 - Lung | − | N/A |
| | 2F 38 - Injection site | ++ | − |
| | 2F 39 - Lung | − | N/A |
| | 2F 39 - Injection site | − | N/A |
| | 2F 50 - Lung | − | N/A |
| | 2F 50 - Injection site | − | N/A |
| ABCB5- | 3M 11 - Lung | − | N/A |
| positive | 3M 11 - Injection site | − | N/A |
| cells - | 3M 12 - Lung | − | N/A |
| 4 weeks | 3M 12 - Injection site | − | N/A |
| | 3M 13 - Lung | − | N/A |
| | 3M 13 - Injection site | − | N/A |
| | 3M 14 - Lung | − | N/A |
| | 3M 14 - Injection site | (a) | N/A |
| | 3M 15 - Lung | − | N/A |
| | 3M 15 - Injection site | − | N/A |
| | 3F 40 - Lung | − | N/A |
| | 3F 40 - Injection site | − | N/A |
| | 3F 41 - Lung | − * | N/A |
| | 3F 41 - Injection site | − | N/A |
| | 3F 42 - Lung | − | N/A |
| | 3F 42 - Injection site | − | N/A |
| | 3F 43 - Lung | − | N/A |
| | 3F 43 - Injection site | − | N/A |
| | 3F 51 - Lung | − | N/A |
| | 3F 51 - Injection site | − | N/A |
| ABCB5- | 4M 16 - Lung | (b) | N/A |
| positive | 4M 16 - Injection site | − | N/A |
| cells - | 4M 17 - Lung | − | N/A |
| 13 weeks | 4M 17 - Injection site | N/A | N/A |
| | 4M 18 - Lung | − | N/A |
| | 4M 18 - Injection site | N/A | N/A |
| | 4M 19 - Lung | − | N/A |
| | 4M 19 - Injection site | − | N/A |
| | 4M 20 - Lung | − | N/A |
| | 4M 20 - Injection site | N/A | N/A |
| | 4F 45 - Lung | − | N/A |
| | 4F 45 - Injection site | N/A | N/A |
| | 4F 46 - Lung | − | N/A |
| | 4F 46 - Injection site | − | N/A |
| | 4F 47 - Lung | − | N/A |
| | 4F 47 - Injection site | N/A | N/A |
| | 4F 48 - Lung | − | N/A |
| | 4F 48 - Injection site | − | N/A |
| | 4F 49 - Lung | − | N/A |
| | 4F 49 - Injection site | − | N/A |

− = Negative;
+ Weak positive;
++ Moderately positive;
+++ Strongly positive;
N/A = Not Applicable
* Result obtained in separate run
(a) Subcutaneous tissues cannot be adequately assessed due to section damage.
(b) Weak cytoplasmic staining in a focal area of tissue adjacent to the lung in levels 2 and 3. The cells do not resemble mesenchymal cells observed in other sections and are considered to represent non-specific background staining.

A total of 30 lung samples and 25 skin samples were analyzed in this study, and only one positive animal was found for each region. Both positive findings were obtained in animals that were sacrificed one week after cell injection and no human cells could be detected with this method at later time points (see Table 4). The advantage of histological staining is that actual cells can be visualized and analyzed for specific markers. It was possible to prove the existence of human cells one week after cell administration in two animals, which was expected in the highly immune compromised NSG mice. Entrapment of MSCs in the lungs after intravenous administration is described in the literature in preclinical studies (Sensebe and Fleury-Cappellesso 2013; Wang et al. 2015; Leibacher and Henschler 2016) and was also observed after MSC administration into humans (Gholamrezanezhad et al. 2011) and is thus not surprising. Cells detected in a thrombus in a blood vessel of the lung of animal 2F 36 were in a highly active microenvironment, which is a likely explanation that there may still be some Ki67 positive cells among them. The cells were scattered over the investigated areas and did not form clusters, and the pathological examination of the toxicology/tumorigenicity study did not reveal any signs of tumor formation even after three bi-weekly cell applications. The results of this study confirm that in NSG mice cells can persist in lungs (n=1) and at the injection site (n=1) for at least a week and a small number of the remaining cells may still be proliferative (n=1). There were no signs of cluster formation and no cells could be detected anymore at the 4 or 13 weeks timepoints.

### Example 3

### Toxicology and Tumorigenicity

The toxicity and tumorigenic potential of human ABCB5-positive Mesenchymal Stem Cells (MSCs), in NOD/SCID/IL2Rγ^{null} (NSG) mice after 3 bi-weekly intravenous injections were examined. Mice were observed for 13 weeks.

Immune-compromised NOD/SCID/IL2Rγ^{null} (NSG) mice at 7-8 weeks of age (n=10/sex/group) were treated either with vehicle (HRG; HSA, Lactated Ringer's solution, Glucose) or with ABCB5-positive cells in vehicle at doses of 2×10⁶ cells by i.v. injection into the left or right caudal veins with a 26G needle at a volume of 200 μl. Animals were treated three times (on Days 1, 15 and 29) and were monitored for 13 weeks. HeLa cells were used as positive control and applied to a separate group of mice (n=5/sex/group) by s.c. injection. The design for the study to investigate the tumorigenic potential of ABCB-positive MSCs was based on respective guidance documents, also including the WHO's “Recommendations for the evaluation of animal cell cultures as substrates for the manufacture of biological medicinal products and for the characterization of cell banks” (2010). As explained in Chapter B.8 therein, the positive control was chosen to show that in the animal model tumor growth can occur and that tumors can be detected. The application route of the positive control cell line does not need to be the same as the clinical route for the test drug product. HeLa cells are a very commonly used cell line for tumor development in mice and are recommended by the WHO guidance document. For this cell line, subcutaneous application is recommended. Deviating from the recommendation of the WHO only a cell dose of 1×10⁶ cells/animal was be administered. This dose however was expected to be sufficient for tumor development in NSG mice.

TABLE 5
Experimental Groups and Doses

| | | | | | ABCB5- | | |
|---|---|---|---|---|---|---|---|
| | | | | | positive | | |
| | Number | | | | MSC | | |
| | of | | | Observation | Suspension | Admin. | Dose Level |
| Group | Animals | Treatment | Route | Period | [cells/ml] | Volume | [cells/animal] |
| 1* | 10 M | Vehicle | i.v. | 13 weeks | | 200 μl | |
| | 10 F | | | | | | |
| 2* | 10 M | ABCB5- | i.v. | 13 weeks | 1 × 10⁷ | 200 μl | 2 × 10⁶ |
| | 10 F | positive | | | | | |
| | | cells | | | | | |
| 3 | 5 M | HeLa cells | s.c. | 13 weeks | 5.4 × 10⁶ | 200 μl | 1.08 × 10⁶ |
| | 5 F | (positive | | | | | |
| | | control) | | | | | |

* Dosing was repeated three times (on Days 1, 15 and 29) resulting in a total cumulative Dose of 6 × 10⁶ cells per animal for Group 2 animals.

During the study animals were monitored regarding mortality, clinical parameters and ophthalmology as well as body weight, food consumption and laboratory examinations like hematology, blood chemistry and the palpation of tumors. After necropsy macropathology was undertaken, organs were weighed and examined regarding histopathological parameters. To determine the tumorigenic potential of ABCB5-positive cells, mass formation was palpated 3 times a week for the first four weeks and weekly thereafter.
Mortality: One animal dosed with the test item was found dead after dosing on Day 1 and was replaced, one animal dosed with the test item was found dead after dosing on Day 15. Both these animals had no macroscopic abnormalities and their deaths were considered to be due to the administration procedure. One animal of the positive-control group developed a carcinoma and was killed for welfare reasons on Day 63 due to chewing and scratching an ulcerated mass.
Clinical Signs and Palpation: There were no test item-related clinical signs. Procedural-related bruising was observed at the intravenous injection sites for all vehicle control and test item-treated animals. No palpable masses were detected on any vehicle control or test item-treated animals. For all positive control mice dorsocranial and/or dorsocaudal masses were observed on the right side in the region of the injection site. Three mice also had dorsocranial/caudal masses on the left side.
Body weight: Body weight development for male and female mice is shown in FIG. 10. It can be seen that in male mice a difference developed between treatment groups at the beginning of the study which was not worsening throughout the study. In total there was a difference in body weight development at the end of the observation period of 13 weeks, because the control animals gained 6.5 g and the cell-treated male animals 4.7 g during this period. A comparison of the body weight at week 13 showed a small difference in body weight of male mice of about 5%, which is considered to be of minor relevance by the CRO Envigo and the toxicological consultants of the sponsor. The slight difference was related to reduced food intake of the affected animals. No effect on body weight was seen in body weight development of females.
Hematology and blood chemistry: Some minor test item-related changes comprised high white blood cell values due to high monocytes counts (males), low blood glucose (females), high potassium (females) and low cholesterol and triglyceride values and liver weights (both sexes). Positive control mice showed several differences from vehicle controls in the parameters determined for haematology and blood chemistry and can be found in detail in the study report.
Macropathology and Histopathology: After 3 bi-weekly intravenous injections of human ABCB5-positive MSCs, there were no test-item related changes in macropathology or histopathology. For positive control animals macropathology revealed palpable masses and enlarged spleens and carcinomas were observed in all (10/10) positive control animals with HeLa cells by subcutaneous injection.
Conclusion: It is concluded that 3 bi-weekly intravenous injections of ABCB5-positive Mesenchymal Stem Cells to NOD/SCID/IL2R₆₅ ^{null} mice at 2×10⁶ cells/animal with a 13-week observation period was well tolerated with no signs of tumorigenicity or findings of toxicological significance. The dose of up to 2×10⁶ human ABCB5-positive cells is considered as No Observed Adverse Effect Level (NOAEL).
Minor test item-related changes comprised low body weight gain (males), high white blood cell values due to high monocytes counts (males), low blood glucose (females), high potassium (females) and low cholesterol and triglyceride values and liver weights (both sexes). Masses were present in all positive control animals, demonstrating the capacity of this strain to develop tumors.

### Immunostaining of Mouse Tissue

The purpose of this study was to evaluate the presence and proliferation status of remaining ABCB5-positive Mesenchymal Stem Cells (MSCs) in lungs and injection sites derived from toxicity and tumorigenicity study (3 bi-weekly intravenous injections with a 13-week observation period).

Formalin-Fixed, Paraffin Embedded (FFPE) blocks of mouse tissue were generated in the course of the study described above and transferred to this study. Tissues of 4 control vehicle animals, all 20 animals treated with ABCB5-positive cells and 1 animal treated with HeLA cells were selected for histological analysis. Embedded tissues were sectioned at three levels approximately 100 p.m apart. At each level three sequential sections 4-5 p.m in thickness were taken, one for Haematoxylin and Eosin (H&E) staining—to aid histopathological examination—another for immunohistochemistry using an anti-mitochondrial antibody (AMA). If the AMA-staining was positive (i.e. detected human cells), the corresponding third slide was then stained with Ki67 antibody. Table 1 lists all tissue samples that were used for the immunostaining.

TABLE 1
Animal tissues used for immunostaining

| | | | | Histological analysis | |
|---|---|---|---|---|---|
| | Number | | | Lung tissues | Skin tissues |
| Group | of | | Observation | from (animal | from (animal |
| No. | Animals | Treatment | Period | no.) | no.) |
| 1 | 10 M | Vehicle | 13 weeks | 2 M (3, 9) | — |
| | 10 F | | | 2 F (108, 110) | — |
| 2 | 10 M | ABCB5- | 13 weeks | 10 M | 10 M |
| | | positive | | | |
| | | cells | | | |
| | 10 F | | | 10 F | 10 F |
| 3 | 5 M | HeLa cells | 13 weeks | 1 M (21) | 1 M (21) |
| | | (positive | | | |
| | | control) | | | |
| | 5 F | | | — | — |

Staining with anti-mitochondrial antibody (AMA): Skin and muscle around the injection site (tail section) was investigated from all 20 animals that received ABCB5-positive cells and no positive staining was detected in any animal.

Positive staining for anti-mitochondrial Antibody (AMA) was seen in the lungs of 7 of the 20 animals, indicating the presence of cells of human origin as expected in NSG mice. Staining was exceptionally rare (frequently consisting of no more than 1-2 cells per tissue section) and positively staining cells were situated mostly within the walls of the alveoli or occasionally free within the alveoli, data not shown. Where positive staining was observed, it tended to be present in all three levels. It is considered likely that these cells represent ABCB5-positive mesenchymal stem cells that have persisted in the lungs of these animals. In one animal (2M 14) a cluster of positive staining cells was seen within the lumen of a large vessel in the lungs. This cluster of cells was also visible on the corresponding H&E stained section although the cell type could not be identified. This appeared to be a thrombus that has detached from the vessel wall during histological processing as a small section of vessel wall could be seen adhering to these cells.
Staining with Ki67 antibody: No positive staining for Ki67 was seen that corresponded to the AMA positive cells in any animal, including the cluster of cells found in animal 2M 14. This indicates that although the ABCB5-positive mesenchymal stem cells persisted in the lungs of these animals they were not actively proliferating. These results further confirmed, that the existence of low numbers of cells after 92 days in the severely immunocompromised NSG mouse model is not critical.

Results are listed in Table 7.

TABLE 7
Results of immunostaining of tissues

| Group | Sample ID | AMA Staining | Ki67 Staining |
|---|---|---|---|
| Vehicle | 1M 3 - Lung | − | N/A |
| | 1M 9 - Lung | − | N/A |
| | 1F 108 - Lung | − | N/A |
| | 1F 110 - Lung | − | N/A |
| ABCB5- | 2M 11 - Lung | ++(Scattered alveolar cells) | − |
| positive | 2M 11 - Injection site | − | N/A |
| cells | 2M 12 - Lung | ++(Scattered alveolar cells) | − |
| | 2M 12 - Injection site | − | N/A |
| | 2M 13 - Lung | ++(Scattered alveolar cells) | |
| | 2M 13 - Injection site | − | N/A |
| | 2M 14 - Lung | ++(Intravascular cluster of cells | − |
| | | and scattered alveolar cells) | |
| | 2M 14 - Injection site | − | N/A |
| | 2M 15 - Lung | − | N/A |
| | 2M 15 - Injection site | − | N/A |
| | 2M 16 - Lung | − | N/A |
| | 2M 16 - Injection site | − | N/A |
| | 2M 18 - Lung | − | N/A |
| | 2M 18 - Injection site | − | N/A |
| | 2M 19 - Lung | − | N/A |
| | 2M 19 - Injection site | − | N/A |
| | 2M 20 - Lung | − | N/A |
| | 2M 20 - Injection site | − | N/A |
| | 2M 26 - Lung | ++(Scattered alveolar cells) | − |
| | 2M 26 - Injection site | − | N/A |
| | 2F 111 - Lung | ++(Scattered alveolar cells) | − |
| | 2F 111 - Injection site | − | N/A |
| | 2F 112 - Lung | − | N/A |
| | 2F 112 - Injection site | − | N/A |
| | 2F 113 - Lung | − | N/A |
| | 2F 113 - Injection site | − | N/A |
| | 2F 114 - Lung | − | N/A |
| | 2F 114 - Injection site | − | N/A |
| | 2F 115 - Lung | − | N/A |
| | 2F 115 - Injection site | − | N/A |
| | 2F 116 - Lung | − | N/A |
| | 2F 116 - Injection site | − | N/A |
| | 2F 117 - Lung | − | N/A |
| | 2F 117 - Injection site | − | N/A |
| | 2F 118 - Lung | − | N/A |
| | 2F 118 - Injection site | − | N/A |
| | 2F 119 - Lung | − | N/A |
| | 2F 119 - Injection site | − | N/A |
| | 2F 120 - Lung | ++(Scattered alveolar cells) | − |
| | 2F 120 - Injection site | − | N/A |

− = Negative;
+ Weak positive;
++ Moderately positive;
+++ Strongly positive;
N/A = Not Applicable

Lungs and tail sections (skin and muscle tissue around injection site) of all 20 cell-treated animals of the toxicity and tumorigenicity study were investigated. No cells could be detected in the injection site, but human cells were detected in lungs of 7 animals. This confirms the finding obtained in the biodistribution study, that in NSG mice several weeks after administration the cells are still persisting. Importantly, the immunostaining shows that human cells are not actively proliferating anymore and thus confirms the safety of the cells. Of note, the experimental settings in the toxicity and tumorigenicity study were different from the biodistribution study, as cells were administered three times in bi-weekly intervals and results. This results in a higher total number of cells used and a shorter time between last cell administration and necropsy of the animals.

### Example 4

### Phase IIIA Study and Selection of a Safe Human Starting Dose

The clinical trial will consist of a screening, treatment and efficacy follow-up period, and a safety follow-up period. The subject will be screened, and then the investigational medicinal product (IMP) allo-APZ2-Covid19 will be administered on days 0, 2, and 4. Efficacy will be measured from days 0 to 28, and safety will be monitored from day 0 to month 6.

The aim of this clinical trial is to investigate the efficacy (by general improvement of clinical symptoms such as fever (<37.5° C.), respiratory rate (<24/min without oxygen support), SpO2 (>94% without oxygen support)) and safety (by monitoring adverse events [AEs]) of three doses of the investigational medicinal product (IMP) allo-APZ2-Covid19 administered intravenously to patients suffering from severe COVID-19.

The intended cell dose is 100×10⁶ cells/treatment administered intravenously at three treatment days (Day 0, day 2 and day 4). The flow rate of administration will be 1-2 ml/min. Infusion of the product via a central venous catheter (CVC), a Port-a-Cath (Port) or a similar catheter is also possible. Premedication with antihistamine (at the discretion of the investigator) prior IMP administration to avoid allergic reactions is permitted. Allo-APZ2-Covid19 will be in a concentration of 1×10⁷ cells/mL in HRG-solution. As this is a first-in-human clinical trial, the benefits and risks of allo-APZ2-Covid19 treatment in COVID-19 patients have not yet been investigated. The evaluation of efficacy along with monitoring the incidence of adverse events and serious adverse events are primary objectives of this Phase I/IIa study. However, the efficacy and potential risks of the allo-APZ2-Covid19 has been adequately analyzed in non-clinical studies and clinical trials (see, e.g., Examples 1-3).

The study will enroll male or female patients, ages 18-85 years of age, having a laboratory confirmation of SARS-CoV-2 infection by reverse-transcription polymerase chain reaction (RT-PCR) from any diagnostic sampling source. The subject must have at least one of the following symptoms: dyspnea (RR≥30 breaths/min), pulse oxygen saturation (SpO2)≤93% without oxygen inhalation in resting state, arterial oxygen partial pressure (PaO2)/fraction of inspired oxygen absorption concentration (FiO2)≤300 mmHG, pulmonary imaging showing that the lesion progressed>50% within 24-48 hours, and the patients were managed as severe. The subject also must have adequate renal (CrCl≥30 cc/min) and liver (AST/ALT≤5× ULN) function. Women of childbearing potential must have a negative blood pregnancy test at screening. The exclusion criteria are as follows: life expectancy of <48 hours from screening (at the discretion of the investigator), active malignancy, any known allergies to components of drug IMP and the premedication with antihistamine, current or previous (within 30 days of enrollment) treatment with another investigative drug, or participation and/or under follow-up in another clinical trial, patients anticipated to be unwilling or unable to comply with the requirements of the protocol, evidence of any other medical conditions (such as psychiatric illness, physical examination, or laboratory findings) that may interfere with the planned treatment, affect the patient's compliance, or place the patient at high risk of complications related to the treatment, pregnant or nursing women, and employees of the sponsor, or employees or relatives of the investigator.

The primary efficacy endpoint is the general improvement of clinical symptoms such as fever (<37.5° C.), respiratory rate (<24/min without oxygen support), and/or SpO2 (>94% without oxygen support). The secondary efficacy endpoints include: duration of the initial hospital stay, duration of initial intensive care stay, duration of Oxygen therapy, duration until therapy failure (death or ventilation), and lab values: CRP, Ferritin, TFSG, IL-6, CD4/CD8 counts, lymphocyte count.

The primary safety endpoint is an adverse event, and the secondary safety endpoinst are: physical examination and vital signs at Day 28, and overall survival at Day 28 and at Month 6.

With regards to safety, the biodistribution and persistence of the ABCB5-positive cells were studied in NSG mice (n=5/sex/group) by a single intravenous application at a cell dose of 2×10⁶ cells. Mice were observed for 1 week, 4 weeks or 13 weeks for clinical signs and tissues were investigated by PCR to detect potential human DNA fragments, originating from injected ABCB5-positive cells. The cell application was well tolerated. Investigation of the tissue distribution showed that quantifiable levels were generally confined to the lungs and injection site tissues up to the end of the study with significant reduction on Day 92.

Furthermore, a combined 13-week repeated dose toxicity and tumorigenicity study was performed in NSG mice. One aim of this GLP study was to provide information on general toxicity to support the route of administration. A second aim of this study was to investigate the tumorigenic potential by determining treatment-induced tumors or metastasis. Therefore, mice (n=10/sex/group+5/sex/HeLa group) were treated three times bi-weekly using intravenous injections of 2×10⁶ ABCB5-positive cells per mouse. In addition to the standard toxicological profile including histopathological examination, potential tumor formation was monitored by palpation during the course of the study. No test item related mortality and adverse effects were noted, considering the investigated parameters: clinical findings, body weight and food consumption, hematology, clinical chemistry, coagulation, organ weights, macroscopic and histopathological findings. The IMP was well tolerated with no signs of tumorigenicity or findings of toxicological significance.

Taken together, in the safety studies to evaluate intravenous administration of the ABCB5-positive cells for 13 weeks no signs of tumorigenicity or findings of toxicological significance after three bi-weekly administrations were found and cells were not distributed unexpectedly after single dose administration. Sparse amounts of human DNA were found in the biodistribution study after 92 days by qPCR in some animals. With histological analysis some scattered human cells could be detected in 2 of 55 tissues (30 lung samples and 25 skin samples). Both positive findings were obtained in animals that were sacrificed one week after cell injection and no human cells could be detected with this method at later time points. Tissue obtained from the tumorigenicity and toxicology was analyzed histologically as well, and 9 weeks after the third cell application cell were still detected in lung tissue of 35% of all animals but none of these cells were positive for the proliferation marker Ki67. It is therefore concluded that in the chosen animal model ABCB5-positive cells are initially persisting but show long-term degradation. The GLP-safety studies showed that cell treatment was well tolerated and revealed no safety concerns.

It is anticipated that there will be a benefit for COVID-19 patients upon treatment with allo-APZ2-Covid19 based on the results of the nonclinical studies. In vitro it was shown that allo-APZ2-Covid19 possess a variety of anti-inflammatory and immunomodulatory properties. Investigations suggest that administered ABCB5-positive cells express anti-inflammatory cytokines and thereby trigger the switch from pro-inflammatory M1 macrophages towards anti-inflammatory M2 macrophages. The anti-inflammatory effect is mediated by IL-1RA, an important molecule responsible for anti-inflammatory suppression of TNF-α in macrophages. In vitro co-culture experiments with macrophages and ABCB5-positive cells confirm this hypothesis and the IL1-RA secretion after co-cultivation of allo-APZ2 with M1 polarized macrophages is also used as a potency assay for the batch release.

Furthermore, the preclinical studies revealed positive effects of intravenously administered ABCB5-positive cells by prolonged cardiac allograft survival and prolonged survival of new-born RDEB-mice (Webber et al. 2017b), as well as improvements in kidney-damage rat model and liver damage mouse models. Recently it was shown that ABCB5 identifies programmed cell death 1 (PD-1) positive Immunoregulatory Dermal Cells (DIRCs) (Schatton et al. 2015). PD-1 is co-expressed with ABCB5 and these cells suppress T-cell proliferation and induce Tregs. Tregs inhibit proinflammatory properties of macrophages and can therefore suppress inflammation (Schatton et al. 2015), which could be vital for the survival of COVID-19 patients.

Therefore, infusion of allo-APZ2-Covid19 while using doses of 100×10⁶ cell/treatment appears to be free of major hazardous events. The planned dose is factor>100 lower than the dose used in the i.v. safety studies (NOAEL).

Moreover, systemic administration of allo-APZ2-Covid19 has been already performed in a clinical setting and demonstrated the safety and tolerability of the product.

In an ongoing phase I/IIa clinical trial for the treatment of epidermolysis bullosa (EB), 16 patients aged between 4 and 36 years old were treated with 3 doses of the IMP intravenously administrated biweekly. Up to date, 3 AEs were classified as possibly related: one patient (36 years old) reported increased lymph nodes which appeared 49 days after the last drug treatment, while two patients (17 and 4 years old) experienced an allergic reaction during the infusion of the 2nd IMP dose, about 2 minutes after the start of the infusion. The patients recovered without sequela after treatment with antihistamine. A data monitoring committee, composed by experts in the field and responsible for monitoring this trial on an ongoing basis, has evaluated this specific adverse event, which was considered to be expected in cell-based drugs. The experts recommended premedication with antihistamine prior to drug administration to avoid such allergic reactions. Therefore, for the purpose of expedited and safety reporting, hypersensitivity events are now considered to be expected.

In an ongoing phase I clinical trial for the treatment of acute-chronic liver failure ACLF (allo-APZ2-ACLF), one patient (34 years old) was treated with 3 doses of the IMP injected on day 0, day 4 and day 11. The patient died 19 days after the last IMP administration; a specific data monitoring committee evaluated this AE that was classified as unrelated to treatment.

Taking the experience of the ongoing EB and ACLF trial together, the drug showed to be safe and well tolerated when intravenously injected, both biweekly and at shorter intervals. Accordingly, the benefit-risk assessment for the use of the drug in a first-in-human Phase I/IIa study in severe COVID-19 patients is positive.

### REFERENCES

Ades, L., P. Guardiola, and G. Socie. 2002. ‘Second malignancies after allogeneic hematopoietic stem cell transplantation: new insight and current problems’, Blood Rev, 16: 135-46.
Amado, L. C., A. P. Saliaris, K. H. Schuleri, M. St John, J. S. Xie, S. Cattaneo, D. J. Durand, T. Fitton, J. Q. Kuang, G. Stewart, S. Lehrke, W. W. Baumgartner, B. J. Martin, A. W. Heldman, and J. M. Hare. 2005. ‘Cardiac repair with intramyocardial injection of allogeneic mesenchymal stem cells after myocardial infarction’, Proc Natl Acad Sci U S A, 102: 11474-9.
Amado, L. C., K. H. Schuleri, A. P. Saliaris, A. J. Boyle, R. Helm, B. Oskouei, M. Centola, V. Eneboe, R. Young, J. A. Lima, A. C. Lardo, A. W. Heldman, and J. M. Hare. 2006. ‘Multimodality noninvasive imaging demonstrates in vivo cardiac regeneration after mesenchymal stem cell therapy’, J Am Coll Cardiol, 48: 2116-24.
Ankrum, J. A., J. F. Ong, and J. M. Karp. 2014. ‘Mesenchymal stem cells immune evasive, not immune privileged’, Nat Biotechnol, 32: 252-60.
Baraniak, P. R., and T. C. McDevitt. 2010. ‘Stem cell paracrine actions and tissue regeneration’, Regen Med, 5: 121-43.
Barkholt, L., E. Flory, V. Jekerle, S. Lucas-Samuel, P. Ahnert, L. Bisset, D. Buscher, W. Fibbe, A. Foussat, M. Kwa, O. Lantz, R. Maciulaitis, T. Palomaki, C. K. Schneider, L. Sensebe, G. Tachdjian, K. Tarte, L. Tosca, and P. Salmikangas. 2013. ‘Risk of tumorigenicity in mesenchymal stromal cell-based therapies-Bridging scientific observations and regulatory viewpoints’, Cytotherapy, 15: 753-9.
Bartsch, G., J. J. Yoo, P. De Coppi, M. M. Siddiqui, G. Schuch, H. G. Pohl, J. Fuhr, L. Perin, S. Soker, and A. Atala. 2005. ‘Propagation, expansion, and multilineage differentiation of human somatic stem cells from dermal progenitors’, Stem Cells Dev, 14: 337-48.
Battula, V. L., P. M. Bareiss, S. Treml, S. Conrad, I. Albert, S. Hojak, H. Abele, B. Schewe, L. Just, T. Skutella, and H. J. Buhring. 2007. ‘Human placenta and bone marrow derived MSC cultured in serum-free, b-FGF-containing medium express cell surface frizzled-9 and SSEA-4 and give rise to multilineage differentiation’, Differentiation, 75: 279-91.
Cao, X., E. W. Shores, J. Hu-Li, M. R. Anver, B. L. Kelsall, S. M. Russell, J. Drago, M. Noguchi, A. Grinberg, E. T. Bloom, and et al. 1995. ‘Defective lymphoid development in mice lacking expression of the common cytokine receptor gamma chain’, Immunity, 2: 223-38.
Cascella, M., M. Rajnik, and A. Cuomo. 2020. Features, Evaluation and Treatment Coronavirus (COVID-19) StatPearls [Internet]. Treasure Island (FL): StatPearls Publishing:.
Chen, S. L., W. W. Fang, F. Ye, Y. H. Liu, J. Qian, S. J. Shan, J. J. Zhang, R. Z. Chunhua, L. M. Liao, S. Lin, and J. P. Sun. 2004. ‘Effect on left ventricular function of intracoronary transplantation of autologous bone marrow mesenchymal stem cell in patients with acute myocardial infarction’, Am J Cardiol, 94: 92-5.
Conti, P., G. Ronconi, A. Caraffa, C. E. Gallenga, R. Ross, I. Frydas, and S. K. Kritas. 2020. ‘Induction of pro-inflammatory cytokines (IL-1 and IL-6) and lung inflammation by Coronavirus-19 (COVI-19 or SARS-CoV-2): anti-inflammatory strategies’, J Biol Regul Homeost Agents, 34.
Dominici, M., K. Le Blanc, I. Mueller, I. Slaper-Cortenbach, F. Marini, D. Krause, R. Deans, A. Keating, Dj Prockop, and E. Horwitz. 2006 ‘Minimal criteria for defining multipotent mesenchymal stromal cells. The International Society for Cellular Therapy position statement’, Cytotherapy, 8: 315-7.
Gang, E. J., D. Bosnakovski, C. A. Figueiredo, J. W. Visser, and R. C. Perlingeiro. 2007. ‘SSEA-4 identifies mesenchymal stem cells from bone marrow’, Blood, 109: 1743-51.
Gholamrezanezhad, A., S. Mirpour, M. Bagheri, M. Mohamadnejad, K. Alimoghaddam, L. Abdolahzadeh, M. Saghari, and R. Malekzadeh. 2011. ‘In vivo tracking of 111In-oxine labeled mesenchymal stem cells following infusion in patients with advanced cirrhosis’, Nucl Med Biol, 38: 961-7.
Halkos, M. E., Z. Q. Zhao, F. Kerendi, N. P. Wang, R. Jiang, L. S. Schmarkey, B. J. Martin, A. A. Quyyumi, W. L. Few, H. Kin, R. A. Guyton, and J. Vinten-Johansen. 2008. ‘Intravenous infusion of mesenchymal stem cells enhances regional perfusion and improves ventricular function in a porcine model of myocardial infarction’, Basic Res Cardiol, 103: 525-36.
Hamming, I., W. Timens, M. L. Bulthuis, A. T. Lely, G. Navis, and H. van Goor. 2004. ‘Tissue distribution of ACE2 protein, the functional receptor for SARS coronavirus. A first step in understanding SARS pathogenesis’, J Pathol, 203: 631-7.
Hare, J. M., J. H. Traverse, T. D. Henry, N. Dib, R. K. Strumpf, S. P. Schulman, G. Gerstenblith, A. N. DeMaria, A. E. Denktas, R. S. Gammon, J. B. Hermiller, Jr., M. A. Reisman, G. L. Schaer, and W. Sherman. 2009. ‘A randomized, double-blind, placebo-controlled, dose-escalation study of intravenous adult human mesenchymal stem cells (prochymal) after acute myocardial infarction’, J Am Coll Cardiol, 54: 2277-86.
Harrell, C. R., C. Fellabaum, N. Jovicic, V. Djonov, N. Arsenijevic, and V. Volarevic. 2019. ‘Molecular Mechanisms Responsible for Therapeutic Potential of Mesenchymal Stem Cell-Derived Secretome’, Cells, 8.
Hartwig, V., B. Dewidar, T. Lin, A. Dropmann, C. Ganss, M. A. Kluth, N. Tappenbeck, L. Tietze, B. Christ, M. H. Frank, R. Vogelmann, M. P. A. Ebert, and S. Dooley. 2019.‘Correction to: Human skin-derived ABCB5(+) stem cell injection improves liver disease parameters in Mdr2KO mice’, Arch Toxicol, 93: 3669-70.
Hashemi, S. M., S. Ghods, F. D. Kolodgie, K. Parcham-Azad, M. Keane, D Hamamdzic, R. Young, M. K. Rippy, R. Virmani, H. Litt, and R. L. Wilensky. 2008. ‘A placebo controlled, dose-ranging, safety study of allogenic mesenchymal stem cells injected by endomyocardial delivery after an acute myocardial infarction’, Eur Heart J, 29: 251-9.
Hatzistergos, K. E., H. Quevedo, B. N. Oskouei, Q. Hu, G. S. Feigenbaum, I. S. Margitich, R. Mazhari, A. J. Boyle, J. P. Zambrano, J. E. Rodriguez, R. Dulce, P. M. Pattany, D. Valdes, C. Revilla, A. W. Heldman, I. McNiece, and J. M. Hare. 2010. ‘Bone marrow mesenchymal stem cells stimulate cardiac stem cell proliferation and differentiation’, Circ Res, 107: 913-22.
Henderson, J. K., J. S. Draper, H. S. Baillie, S. Fishel, J. A. Thomson, H. Moore, and P. W. Andrews. 2002. ‘Preimplantation human embryos and embryonic stem cells show comparable expression of stage-specific embryonic antigens’, Stem Cells, 20: 329-37.
Hoogduijn, M. J., F. Popp, R. Verbeek, M. Masoodi, A. Nicolaou, C. Baan, and M. H. Dahlke. 2010. ‘The immunomodulatory properties of mesenchymal stem cells and their use for immunotherapy’, Int Immunopharmacol, 10: 1496-500.
Huang, C., Y. Wang, X. Li, L. Ren, J. Zhao, Y. Hu, L. Zhang, G. Fan, J. Xu, X. Gu, Z. Cheng, T. Yu, J. Xia, Y. Wei, W. Wu, X. Xie, W. Yin, H. Li, M. Liu, Y. Xiao, H. Gao, L. Guo, J. Xie, G. Wang, R. Jiang, Z. Gao, Q. Jin, J. Wang, and B. Cao. 2020. ‘Clinical features of patients infected with 2019 novel coronavirus in Wuhan, China’, Lancet, 395: 497-506.
Janin, A., H. Murata, C. Leboeuf, J. M. Cayuela, E. Gluckman, L. Legres, A. Desveaux, M. Varna, P. Ratajczak, J. Soulier, H. de The, P. Bertheau, and G. Socie. 2009. ‘Donor-derived oral squamous cell carcinoma after allogeneic bone marrow transplantation’, Blood, 113: 1834-40.
Jiang, D., J. Muschhammer, Y. Qi, A. Kugler, J. C. de Vries, M. Saffarzadeh, A. Sindrilaru, S. V. Beken, M. Wlaschek, M. A. Kluth, C. Ganss, N. Y. Frank, M. H. Frank, K. T. Preissner, and K. Scharffetter-Kochanek. 2016. ‘Suppression of Neutrophil-Mediated Tissue Damage-A Novel Skill of Mesenchymal Stem Cells’, Stem Cells, 34: 2393-406.
Kean, T. J., P. Lin, A. I. Caplan, and J. E. Dennis. 2013. ‘MSCs: Delivery Routes and Engraftment, Cell-Targeting Strategies, and Immune Modulation’, Stem Cells Int, 2013: 732742.
Kebriaei, P., L. Isola, E. Bahceci, K. Holland, S. Rowley, J. McGuirk, M. Devetten, J. Jansen, R. Herzig, M. Schuster, R. Monroy, and J. Uberti. 2009. ‘Adult human mesenchymal stem cells added to corticosteroid therapy for the treatment of acute graft-versus-host disease’, Biol Blood Marrow Transplant, 15: 804-11.
Kim, A J , H. D. Adkisson, M. Wendland, M. Seyedin, S. Berven, and J. C. Lotz. 2010. ‘Juvenile Chondrocytes May Facilitate Disc Repair’, The Open Tissue Engineering and Regenerative Medicine Journal, 3: 28-35.
Kurtzberg, J., S. Prockop, P. Teira, H. Bittencourt, V. Lewis, K. W. Chan, B. Horn, L. Yu, J. A. Talano, E. Nemecek, C. R. Mills, and S. Chaudhury. 2014. ‘Allogeneic human mesenchymal stem cell therapy (remestemcel-L, Prochymal) as a rescue agent for severe refractory acute graft-versus-host disease in pediatric patients’, Biol Blood Marrow Transplant, 20: 229-35.
Leibacher, J., and R. Henschler. 2016. ‘Biodistribution, migration and homing of systemically applied mesenchymal stem/stromal cells’, Stem Cell Res Ther, 7: 7.
Makkar, R. R., M. J. Price, M. Lill, M. Frantzen, K. Takizawa, T. Kleisli, J. Zheng, S. Kar, R. McClelan, T. Miyamota, J. Bick-Forrester, M. C. Fishbein, P. K. Shah, J. S. Forrester, B. Sharifi, P. S. Chen, and M. Qayyum. 2005. ‘Intramyocardial injection of allogenic bone marrow-derived mesenchymal stem cells without immunosuppression preserves cardiac function in a porcine model of myocardial infarction’, J Cardiovasc Pharmacol Ther, 10: 225-33.
Mehta, P., D. F. McAuley, M. Brown, E. Sanchez, R. S. Tattersall, and J. J. Manson. 2020. ‘COVID-19: consider cytokine storm syndromes and immunosuppression’, Lancet.
Muroi, K., K. Miyamura, K. Ohashi, M. Murata, T. Eto, N. Kobayashi, S. Taniguchi, M. Imamura, K. Ando, S. Kato, T. Mori, T. Teshima, M. Mori, and K. Ozawa. 2013. ‘Unrelated allogeneic bone marrow-derived mesenchymal stem cells for steroid-refractory acute graft-versus-host disease: a phase III study’, Int J Hematol, 98: 206-13.
Newman, R. E., D. Yoo, M. A. LeRoux, and A. Danilkovitch-Miagkova. 2009. ‘Treatment of inflammatory diseases with mesenchymal stem cells’, Inflamm Allergy Drug Targets, 8: 110-23.
Ozerdem, U., E. Monosov, and W. B. Stallcup. 2002. ‘NG2 proteoglycan expression by pericytes in pathological microvasculature’, Microvasc Res, 63: 129-34.
Pittenger, M. F., A. M. Mackay, S. C. Beck, R. K. Jaiswal, R. Douglas, J. D. Mosca, M. A. Moorman, D. W. Simonetti, S. Craig, and D. R. Marshak. 1999. ‘Multilineage potential of adult human mesenchymal stem cells’, Science, 284: 143-7.
Poh, K. K., E. Sperry, R. G. Young, T. Freyman, K. G. Barringhaus, and C. A. Thompson. 2007. ‘Repeated direct endomyocardial transplantation of allogeneic mesenchymal stem cells: safety of a high dose, “off-the-shelf”, cellular cardiomyoplasty strategy’, Int J Cardiol, 117: 360-4.
Prasad, V. K., K. G. Lucas, G. I. Kleiner, J. A. Talano, D. Jacobsohn, G. Broadwater, R. Monroy, and J. Kurtzberg. 2011. ‘Efficacy and safety of ex vivo cultured adult human mesenchymal stem cells (Prochymal) in pediatric patients with severe refractory acute graft-versus-host disease in a compassionate use study’, Biol Blood Marrow Transplant, 17: 534-41.
Price, M. J., C. C. Chou, M. Frantzen, T. Miyamoto, S. Kar, S. Lee, P. K. Shah, B. J. Martin, M. Lill, J. S. Forrester, P. S. Chen, and R. R. Makkar. 2006. ‘Intravenous mesenchymal stem cell therapy early after reperfused acute myocardial infarction improves left ventricular function and alters electrophysiologic properties’, Int J Cardiol, 111: 231-9.
Qin, C., L. Zhou, Z. Hu, S. Zhang, S. Yang, Y. Tao, C. Xie, K. Ma, K. Shang, W. Wang, and D. S. Tian. 2020. ‘Dysregulation of immune response in patients with COVID-19 in Wuhan, China’, Clin Infect Dis.
Ra, J. C., I. S. Shin, S. H. Kim, S K. Kang, B. C. Kang, H. Y. Lee, Y. J. Kim, J Y. Jo, E. J. Yoon, H. J. Choi, and E. Kwon. 2011. ‘Safety of intravenous infusion of human adipose tissue-derived mesenchymal stem cells in animals and humans’, Stem Cells Dev, 20: 1297-308.
Rismanbaf, A., and S. Zarei. 2020. ‘Liver and Kidney Injuries in COVID-19 and Their Effects on Drug Therapy; a Letter to Editor’, Arch Acad Emerg Med, 8: e17.
Rothan, H. A., and S. N. Byrareddy. 2020. ‘The epidemiology and pathogenesis of coronavirus disease (COVID-19) outbreak’, J Autoimmun: 102433.
Schatton, T., J. Yang, S. Kleffel, M. Uehara, S. R. Barthel, C. Schlapbach, Q. Zhan, S. Dudeney, H. Mueller, N. Lee, J. C. de Vries, B. Meier, S. Vander Beken, M. A. Kluth, C. Ganss, A. H. Sharpe, A. M. Waaga-Gasser, M. H. Sayegh, R. Abdi, K. Scharffetter-Kochanek, G. F. Murphy, T. S. Kupper, N. Y. Frank, and M. H. Frank. 2015. ‘ABCB5Identifies Immunoregulatory Dermal Cells’, Cell Rep, 12: 1564-74.
Schuleri, K. H., L. C. Amado, A. J. Boyle, M. Centola, A. P. Saliaris, M. R. Gutman, K. E. Hatzistergos, B. N. Oskouei, J. M. Zimmet, R. G. Young, A. W. Heldman, A. C. Lardo, and J. M. Hare. 2008. ‘Early improvement in cardiac tissue perfusion due to mesenchymal stem cells’, Am J Physiol Heart Circ Physiol, 294: H2002-11.
Schuleri, K. H., G. S. Feigenbaum, M. Centola, E. S. Weiss, J. M. Zimmet, J. Turney, J. Kellner, M. M. Zviman, K. E. Hatzistergos, B. Detrick, J. V. Conte, I. McNiece, C. Steenbergen, A. C. Lardo, and J. M. Hare. 2009. ‘Autologous mesenchymal stem cells produce reverse remodelling in chronic ischaemic cardiomyopathy’, Eur Heart J, 30: 2722-32.
Sensebe, L., and S. Fleury-Cappellesso. 2013. ‘Biodistribution of mesenchymal stem/stromal cells in a preclinical setting’, Stem Cells Int, 2013: 678063.
Shi, Y., Y. Wang, C. Shao, J. Huang, J. Gan, X. Huang, E. Bucci, M. Piacentini, G. Ippolito, and G. Melino. 2020. ‘COVID-19 infection: the perspectives on immune responses’, Cell Death Differ.
Silva, G. V., S. Litovsky, J. A. Assad, A. L. Sousa, B. J. Martin, D. Vela, S. C. Coulter, J. Lin, J. Ober, W. K. Vaughn, R. V. Branco, E. M. Oliveira, R. He, Y. J. Geng, J. T. Willerson, and E. C. Perin. 2005. ‘Mesenchymal stem cells differentiate into an endothelial phenotype, enhance vascular density, and improve heart function in a canine chronic ischemia model’, Circulation, 111: 150-6.
Singhal, T. 2020. ‘A Review of Coronavirus Disease-2019 (COVID-19)’, Indian J Pediatr, 87: 281-86.
Vaculik, C., C. Schuster, W. Bauer, N. Iram, K. Pfisterer, G. Kramer, A. Reinisch, D. Strunk, and A. Elbe-Burger. 2012. ‘Human dermis harbors distinct mesenchymal stromal cell subsets’, J Invest Dermatol, 132: 563-74.
Vander Beken, Seppe, Juliane C. de Vries, Barbara Meier-Schiesser, Patrick Meyer, Dongsheng Jiang, Anca Sindrilaru, Filipa F. Ferreira, Adelheid Hainzl, Susanne Schatz, Jana Muschhammer, Natalie J. Scheurmann, Panagiotis Kampilafkos, Andreas M. Seitz, Lutz Dürselen, Anita Ignatius, Mark A. Kluth, Christoph Ganss, Meinhard Wlaschek, Karmveer Singh, Pallab Maity, Natasha Y. Frank, Markus H. Frank, and Karin Scharffetter-Kochanek. 2019. ‘Newly Defined ATP-Binding Cassette Subfamily B Member 5 Positive Dermal Mesenchymal Stem Cells Promote Healing of Chronic Iron-Overload Wounds via Secretion of Interleukin-1 Receptor Antagonist’, Stem Cells, 37: 1057-74.
Wada, N., S. Gronthos, and P. M. Bartold. 2013 ‘Immunomodulatory effects of stem cells’, Periodontol 2000, 63: 198-216.
Wang, F., S. Eid, J. E. Dennis, K. R. Cooke, J. J. Auletta, and Z. Lee. 2015. ‘Route of delivery influences biodistribution of human bone marrow-derived mesenchymal stromal cells following experimental bone marrow transplantation’, J Stem Cells Regen Med, 11: 34-43.
Wang, Y., X. Chen, W. Cao, and Y. Shi. 2014. ‘Plasticity of mesenchymal stem cells in immunomodulation: pathological and therapeutic implications’, Nat Immunol, 15: 1009-16.
Webber, B. R., K. T. O′Connor, R. T. McElmurry, E. N. Durgin, C. R. Eide, C. J. Lees, M. J. Riddle, W. E. Mathews, N. Y. Frank, M. A. Kluth, C. Ganss, B. S. Moriarity, M. H. Frank, M. J. Osborn, and J. Tolar. 2017a. ‘Rapid generation of Col7a1−/− mouse model of recessive dystrophic epidermolysis bullosa and partial rescue via immunosuppressive dermal mesenchymal stem cells’, Lab Invest, 97: 1218-24.
2017b. ‘Rapid generation of Col7a1(-/-) mouse model of recessive dystrophic epidermolysis bullosa and partial rescue via immunosuppressive dermal mesenchymal stem cells’, Lab Invest, 97: 1218-24.
WHO_COVID-19_Situation_Report-65. 2020. ‘WHO Coronavirus disease 2019 (COVID-19) Situation Report —65’.
Williams, A. R., B. Trachtenberg, D. L. Velazquez, I. McNiece, P. Altman, D. Rouy, A. M. Mendizabal, P. M. Pattany, G. A. Lopera, J. Fishman, J. P. Zambrano, A. W. Heldman, and J. M. Hare. 2011. ‘Intramyocardial stem cell injection in patients with ischemic cardiomyopathy: functional recovery and reverse remodeling’, Circ Res, 108: 792-6.
Wolf, D., A. Reinhard, A. Seckinger, H. A. Katus, H. Kuecherer, and A. Hansen. 2009. ‘Dose-dependent effects of intravenous allogeneic mesenchymal stem cells in the infarcted porcine heart’, Stem Cells Dev, 18: 321-9.
Wollert, K. C., G. P. Meyer, J. Lotz, S. Ringes-Lichtenberg, P. Lippolt, C. Breidenbach, S. Fichtner, T. Korte, B. Hornig, D. Messinger, L. Arseniev, B. Hertenstein, A. Ganser, and H. Drexler. 2004. ‘Intracoronary autologous bone-marrow cell transfer after myocardial infarction: the BOOST randomised controlled clinical trial’, Lancet, 364: 141-8.
Xu, L., J. Liu, M. Lu, D. Yang, and X. Zheng. 2020. ‘Liver injury during highly pathogenic human coronavirus infections’, Liver Int.

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method of treating a hyper-inflammatory disorder in a human subject, the method comprising:
administering to the subject a composition comprising ABCB5+ stem cells in an effective amount to treat the hyper-inflammatory disorder.

2. The method of claim 1, wherein the dose is 1×10⁶ to 1×10¹⁰, optionally 1×10⁸ ABCB5+ stem cells.

3. The method of claim 1, further comprising administering the dose to the subject two times.

4. The method of any one of claim 1, wherein the dose is administered to the subject three times.

5. The method of claim 1, wherein the dose is administered to the subject four times.

6. The method of claim 3, wherein the doses are administered one day apart.

7. The method of claim 1, wherein the composition comprises ABCB5+ stem cells and a pharmaceutically acceptable excipient.

8. The method of claim 7, wherein the pharmaceutically acceptable excipient is human serum albumin/Ringer/glucose solution (HRG).

9. The method of claim 1, wherein the inflammatory disorder is acute respiratory distress syndrome (ARDS).

10. The method of claim 1, wherein the subject has a severe COVID-19 infection.

11. The method of claim 1, wherein administration of the dose increases the level of IL-IRA, IL-10, or both, in the subject.

12. The method of claim 1, wherein administration of the dose decreases the level of TNF-α, IL-1β, or both, in the subject.

13. The method of claim 1, wherein administration of the dose promotes a switch from M1 macrophages to M2 macrophages.

14. A method of treating a human subject having a SARS infection, the method comprising:
administering a composition of ABCB5+ stem cells to the subject in an effective amount to treat the subject.

15. The method of claim 14, wherein the SARS infection is a SARS-CoV-2 infection.

16. The method of claim 14, wherein the ABCB5+ stem cells are dermal ABCB5+ stem cells.

17. The method of claim 14, wherein the ABCB5+ stem cells are ocular ABCB5+ stem cells.

18. The method of claim 14, wherein the ABCB5+ stem cells are a population of synthetic ABCB5+ stem cells.

19. The method of claim 18, wherein greater than 99%, 99.5%, 99.7%, 99.9%, 99.99%, 99.998%, 99.999%, or 99.999997% of the population of synthetic ABCB5+ stem cells is an in vitro progeny of physiologically occurring skin-derived ABCB5-positive mesenchymal stem cells.

20. The method of claim 14, wherein the cells are administered intravenously.

21. The method of claim 14, wherein a dose of the cells is 1×10⁶ to 1×10¹⁰ ABCB5+ stem cells.

22. The method of claim 1, wherein administration of the cells increases the level of IL-IRA, IL-10, or both, in the subject.

23. The method of claim 1, wherein administration of the cells decreases the level of TNF-α, IL-1β, or both, in the subject.

24. The method of claim 1, wherein administration of the cells promotes a switch from M1 macrophages to M2 macrophages.
